SEQUENCE LISTING (5 sequences)

SEQ ID NO: 1 - DNA, 12 bp, Artificial Sequence
  Description of Artificial Sequencewild type p53 exon 5 of DNA binding domain
atgttttgccaa

SEQ ID NO: 2 - DNA, 12 bp, Artificial Sequence
  Description of Artificial SequenceRL-7 mutant p53 exon 5 of DNA binding domain (C135F)
atgtttttccaa

SEQ ID NO: 3 - DNA, 12 bp, Artificial Sequence
  Description of Artificial Sequencewild type p53 exon 9
cagccaaagaag

SEQ ID NO: 4 - DNA, 12 bp, Artificial Sequence
  Description of Artificial SequenceK422 mutant p53 exon 9 truncation protein with pre-terminal stop codon at position 319
cagccatagaag

SEQ ID NO: 5 - DNA, 10 bp, Artificial Sequence
  Description of Artificial Sequencep53 consensus element
rrrcwwgyyy

(19) United States
(12) Patent Application Publication
(10) Pub. No.: US 2004/0101915 A1
(43) Pub. Date: May 27, 2004

(54) Diagnosis and treatment of chemoresistant tumors

(75) Inventors: Quinn L. Deveraux, San Diego, CA (US); Klaus W. Wagner, San Diego, CA (US); Garrett M. Hampton, San Diego, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: IRM LLC, a Delaware Limited Liability Company, Hamilton (BM)

(21) Appl. No.: 10/464,996

(22) Filed: Jun. 18, 2003

Related U.S. Application Data
(60) Provisional application No. 60/456,585, filed on Mar. 21, 2003.
(60) Provisional application No. 60/390,256, filed on Jun. 18, 2002.

(51) Int. Cl. C12Q 1/68; G01N 33/574

(52) U.S. Cl. 435/7.23; 435/6

(57) ABSTRACT

This invention provides methods of identifying compounds that selectively target cancer cells that have defects in specific oncogenic pathways.

### CROSS-REFERENCES TO RELATED APPLICATIONS

This application claims benefit of U.S. provisional application No. 60/390,256, filed Jun. 18, 2002, and U.S. provisional application No. 60/456,585, filed Mar. 21, 2003, each of which is incorporated by reference herein.

### FIELD OF THE INVENTION

This invention relates to the field of diagnosis, prognosis, and treatment of chemoresistant cancers, including chemoresistant lymphomas.

### BACKGROUND OF THE INVENTION

Although oncogenesis is a complex multistep process, it has been postulated that the vast differences of cancer genotypes are in principal manifestations of six common and essential capabilities of all human tumors (see, e.g., Hanahan & Weinberg, Cell 100:57-70, 2000). Central among these capabilities is the acquisition of resistance to apoptosis (see, e.g., Evan & Vousden, Nature 411:342-8, 2001). Although numerous mechanisms for cell death resistance have now been elucidated, the first mammalian negative regulator of apoptosis bcl-2 (for B-cell lymphoma) was discovered in tumor cells derived from a follicular lymphoma (FL) patient (see, e.g., Adams & Cory, Science 281:1322-1326, 1998; Bakhshi et al., Cell 41:899-906, 1985; and Tsujimoto et al., Science 228:1440-1443. 1985). This low-grade B-cell Non-Hodgkin's lymphoma is characterized by the chromosome translocation t(14;18)(q32;21), which couples the bcl-2 gene and its expression, to the immunoglobulin heavy chain enhancer. FL is a an indolent slow-growing lymphoma with a median survival time between 8 and 10 years, possibly reflecting bcl-2's role as a survival factor, rather than a pro-mitotic stimulus.

FL patients often achieve a clinical complete response after the first treatment with standard chemotherapeutic regimens; however, the clinical course of the vast majority of cases, particularly in advanced stages III and IV, consists of repeated relapses (see, e.g,, Freedman et al., Oncology (Huntingt) 14:321-326, 329, discussion 330-2, 338, 2000; Horning Ann Oncol 11 Suppl 1:23-27, 2000; and Peterson, Semin Oncol 26:2-11, 1999). Complete or partial remissions can be induced with subsequent therapies, but at a progressively lower rate and with shorter duration. One explanation for the high relapse rate following initial clinical response is the presence of minimal residual disease (MRD). Residual lymphoma cells, which survive chemotherapy, persist below the detection threshold of standard clinical diagnostic procedures, including CT scans, which have a lower sensitivity than molecular methods such as t(14;18)-specific PCR of peripheral blood and/or bone marrow samples. Recent studies employing bcl-2/IgH rearrangement-specific PCRs as a marker for MRD have shown that standard chemotherapy rarely results in PCR negativity. Thus, determination of a molecular response seems to be the most important factor for predicting the failure-free survival of FL patients (see, e.g., Lopez-Guillermo et al., Blood 91:2955-60, 1998).

Two apoptotic pathways can initiate apoptosis: (1) the death receptor (extrinsic) pathway or (2) the mitochondrial dependent (intrinsic) pathway. In both cases, cysteine aspartyl-specific proteases (caspases) are activated that cleave cellular substrates, thereby leading to the biochemical and morphological characteristics of apoptotic cells (see, e.g, Salversen & Dixit, Proc. Natl. Acad Sci USA 96:10964-10967, 1999; Budihardjo et al., Ann. Rev. Cell Dev. Biol. 15:239-90, 1999; and Zimmermann & Green, J. Allergy Clin Immunol. 108:99-103, 2001). Anticancer DNA damaging agents ultimately activate effector caspases such as caspase-3, however, there is controversy concerning which pathways connect the DNA damage response to the activation of caspases (see, e.g., Kaufmann & Earnshaw, Exp Cell Res. 256:42-9, 2000).

Anticancer drugs have been reported to enhance the expression of pro-apoptotic bcl-2 family members such as BAX, PUMA and NOXA (see, e.g., Nakano & Vousden, Mol. Cell Biol. 7:683-94, 2001; Oda et al, Science 288:1053-58, 2000; and Wu & Deng, Front Biosci. 7:151-6, 2002). These pro-apoptotic proteins target the mitochondria promoting release of apoptosis initiating factors, such as cytochrome c (e.g., Huang & Strasser, Cell 103:9357-60, 2000). Once released into the cytoplasm, cytochrome c associates with Apaf-1 and pro-caspase-9 forming a complex termed the “apoptosome”. Activation of pro-caspase-9 occurs within the apoptosome, thereby initiating the activation of effector caspases such as caspase-3 (see, e.g., Li et al., Cell 91:479-489, 1997). Anti-apoptotic members of the bcl-2 family, such as bcl-2 and bcl-X_{L}, can suppress this intrinsic cell death pathway by blocking mitochondrial release of cytochrome c and its association with Apaf-1.

Recently, several reports have suggested that anticancer drugs can kill susceptible tumor cells by inducing the expression of death receptors (CD95, TRAIL-DR4 or TRAIL-DR5) and/or their corresponding ligands (CD95L, TRAIL) (see, e.g., Friesen et al., Leukemia 13:1854-8, 1999; Gibson et al, Mol. Cell Biol. 20:205-12, 2000; Muller et al, J Exp Med 188:2033-45, 1998 and Wen et al., Blood 96:3900-6, 2000). Triggering of death receptors induces recruitment of the adapter molecule FADD and pro-caspase-8, which form the death-inducing signaling complex (DISC) where pro-caspase-8 is activated by autocatalytic cleavage (e.g., Kisckel et al. EMBO J. 14:5579-88, 1995; and Salveson & Sixit, supra). Active caspase-8 is involved in formation of higher-order surface receptor clusters as shown for the CD95 receptor (e.g., Algeciras-Schimnich et al, Mol Cell Biol 22:207-20, 2002) and leads to the proteolytic activation of downstream effector caspases, especially caspase-3. Thus, death receptor pathways can bypass cell death inhibition mediated by overexpressed anti-apoptotic members of the bcl-2 family (e.g., Walczak et al, Cancer Res 60:3051-7, 2000). However, the death receptor or extrinsic pathways and the intrinsic cell death program are not necessarily mutually exclusive. For example, in some cell types, the extrinsic and intrinsic pathways are interconnected by caspase-8-mediated cleavage of the pro-apoptotic bcl-2 family member Bid. Following cleavage, truncated Bid translocates to the mitochondria promoting cyctochrome c release—which can be readily blocked by overexpression of bcl-2 (Scaffidi et al, EMBO J. 17:1675-87, 1998).

Given the versatility of bcl-2 to suppress cell death (see, e.g., Reed, Semin Hematol 34:9-19, 1997; and Simonian et al, Blood 90:1208-16, 1997), it is surprising that cancer patients such as FL patients, whose cancer cells overexpress bcl-2, respond to initial chemotherapeutic treatments. Equally perplexing is that despite initial responses, FL patients exhibit exceptionally high relapse rates. Accordingly, a need exists for a means to identify resistance mechanisms and treat chemoresistant follicular lymphomas. The present invention fulfils these and other needs. Moreover, the methods disclosed herein can be used to identify selective drug therapies for the treatment of other cancers, e.g., other chemoresistant tumors.

### BRIEF SUMMARY OF THE INVENTION

In some embodiments, this invention provides methods of screening for a compound that is selectively cytotoxic to cancer cells, in particular, chemoresistant cancer cells. These methods involve: a) contacting a candidate compound with chemoresistant cancer cells, typically those that have one or more defined defects in a cell death pathway, e.g., a TRAIL-DR5 death receptor pathway; and b) determining whether the candidate compound is cytotoxic to the cancer cells in comparison to normal cells, or in comparison to a population of cancer cells that have one or more different defects in an apoptotic pathway, e.g., a TRAIL-DR5 apoptotic pathway. A candidate agent that is cytotoxic to the cancer cells in the screen is effective against chemoresistant cancer cells.

The invention also provides methods for treating a cancer patient. These methods involve: a) testing cancer cells from a cancer patient for a defect in a TRAIL-DR5 death receptor pathway; and b) if cells exhibit a defect in the TRAIL-DR5 death receptor pathway, treating the cancer with a therapeutic agent for which induction of DNA damage is not the therapeutic agent's primary mechanism of action. Such therapeutic agents include, for example, an anti-CD20 antibody, an allogeneic T-lymphocyte, a bcl-2 inhibitor, an agonistic anti-TRAIL antibody, or a TRAIL receptor ligand. For example, an agonistic anti-DR5 antibody or a ligand for DR5 can be used as the treatment for the chemoresistant cancer, as can rituximab or TRAIL/Apo2L.

In some embodiments, the cancer patient is treated with a DNA damaging agent prior to testing for the p53 defect or the TRAIL-DR5 death receptor pathway defect. For example, the DNA damaging agent can be an alkylating agent, a topoisomerase II inhibitor, or other compounds known to those of skill in the art.

In other embodiments, the invention provides methods for identifying a patient having, or at risk of developing, a chemoresistant cancer, such as a chemoresistant lymphoma. These methods involve testing cancer cells from a patient for a defect in a TRAIL-DR5 death receptor pathway. A defect in the pathway is indicative of a chemoresistant cancer. The chemoresistant cancers can be resistant to, for example, a chemotherapeutic agent selected from the group consisting of an alkylating agent and a topoisomerase II inhibitor. A defect in a TRAIL-DR5 death receptor pathway is indicated by, for example, an inability of a chemotherapeutic agent to induce apoptosis. The defect can be, for example, a defect in p53, a defect in caspase 3 expression, a defect in another component of the death receptor pathway, and the like. The methods are useful for diagnosing and prognosticating for cancers that include, for example, non-Hodgkin's lymphomas such as follicular lymphomas. The cancers for which the methods are suitable include those that overexpress Bcl-2.

In another embodiment, the invention provides methods for monitoring a treatment of a cancer patient. These methods involve testing cancer cells obtained from the patient periodically during a cancer treatment for a defect in a TRAIL-DR5 death receptor pathway. An increase during treatment in prevalence of cancer cells having such a defect is indicative that the cancer cells are becoming resistant to the cancer treatment. Accordingly, in some embodiments, the methods further involves treating the patient with a therapeutic agent for which induction of DNA damage is not the therapeutic agent's primary mechanism of action if a defect in the TRAIL-DR5 death receptor pathway is observed in the cancer cells.

The invention also provides methods for identifying a gene that is involved in a cell death pathway. These methods involve: a) providing a library of cDNA or inhibitory RNA molecules, wherein each library member is present in a cell; b) contacting the cells with a compound that can modulate cell death in a cell that does not comprise the cDNA or inhibitory RNA molecules; and c) identifying library members in which the compound modulates cell death differently than in a cell that does not contain the cDNA or inhibitory RNA molecule.

### BRIEF DESCRIPTION OF THE DRAWINGS

FIG. 1. (A) Differential sensitivity of FL cell lines to anticancer drugs. DOHH2 (▪) Jurkat (●), Jurkat bcl-2 (+), K422 (▴) and RL-7 (□) cells were treated with the indicated concentrations of etoposide and doxorubicin for 24 hours. Cell viability was determined by MTT assays (A) and caspase assays confirmed the drug sensitivity of DOHH2 and resistance of K422 and RL-7 cells (data not shown). The data are the mean of triplicates, similar results were obtained in four independent experiments and also with tubulin inhibitors (e.g., vincristine). (B) FL cell lines are resistant to UV-induced cell-death. The indicated cell lines were exposed to increasing doses of UV light (×100 μJ/cm²) and cell viability was determined by MTT assays, which revealed that the FL cell lines were more resistant to UV induced apoptosis than control Jurkat T cells. Bcl-2 protein levels were confirmed by Western blot analysis using antibodies specific for the bcl-2 protein (upper panel).

FIG. 2. (A) Gene expression analysis of etoposide treated FL cell lines. RNA samples from each FL cell line were prepared following 0, 2 and 4 hours of exposure to 4.25 μM etoposide. Gene expression analysis was performed using U95Av2 arrays (Affymetrix) as previously described (Wodicka et al, Nat Biotechnol. 15: 1359-67:1997). All genes (12,533) were filtered using the following criteria: average difference fold change≧2.0 after 2 or 4 hours of etoposide treatment in one of the three FL cell lines (DOHH2, K422 or RL-7) and maximum hybridization intensity (AD)>200 (in at least one sample: untreated, 2 h or 4 h after etoposide treatment). Data from two independent experiments were averaged. For each gene, which fulfilled these criteria, the average difference fold change across all cell lines was median-centered, normalized and displayed as an increase in red or decrease in blue compared to the median difference fold change. The color saturation is proportional to the magnitude of the difference to the mean. (B) Raw expression data for several, reported p53 regulated genes. Median and standard deviation for 0, 2 and 4 hours following addition of etoposide are the result of two independent experiments. Grey bars DOHH2, white K422 and RL-7 striped bars respectively.

FIG. 3. P53 mutations found in the chemotherapy resistant K422 and RL-7 cell lines and localization within the p53 domain structure. The coding sequences of the p53 gene of DOHH2, K422 and RL-7 cells were amplified with mRNA specific primers, cloned into pCR4-TOPO (Invitogen) and sequenced. The frequency of individual codon mutations as % of single base pair substitutions are illustrated in the upper panel and were generated by comparison with the p53 mutation database of the WHO's International Agency for Research on Cancer (Hernandez-Boussard et al., International Agency for Research on Cancer., Hum Mutat. 14: 1-8, 1999).

FIG. 4. (A) Etoposide enhances anti-DR5 induced apoptosis in DOHH2, but not in K422 and RL-7 cells. The different FL cell lines were sequentially treated with etoposide (0.425 μM, 4 hours) followed by agonistic anti-DR5 antibodies (concentration as indicated in ng/ml, 12 hours) or with drug (0.425 μM etoposide, 4 hours+12 hours untreated) or antibody (4 hours unexposed+12 hours anti-DR5) alone. The percentage of viable cells was determined at the end of 16 hours by MTT assays. (B) Etoposide induces upregulation of DR5 and activation of caspases only in DOHH2 cells. After treatment with 4.25 μM etoposide for the indicated periods of time (in hours) total cell lysates were prepared and normalized for protein concentration. Caspase activation was measured by Western blots (40 μg protein per lane) and confirmed by caspase activity assays (data not shown). Expression of β-actin was used to control equal protein loading and each experiment was repeated twice with etoposide and doxorubicin with similar results. * Denotes an anti-DR5 immuno-reactive product. The lower smaller arrows in the caspase-8 and caspase-3 panels indicate the active subunits of the proteases.

FIG. 5. Downregulation of caspase-3 in the drug resistant RL-7 cell line. (A) (B) Caspase-8 (Casp8) and cytochrome c (Cyt-c) induced caspase activity in cytosolic extracts prepared from DOHH2, K422 and RL-7 cells. After addition of caspase-8 (20 nM) or cytochrome c (10 μM+1 mM dATP) cell lysates were incubated for the indicated times (in minutes) at 37° C. (C8) or 30° C. respectively (cyt-c) and caspase activities were measured by AFC release from DEVD-AFC (A) or Western blot analysis (B). Cell lysates (40 μg per lane) were separated by 10-20% SDS-PAGE and Western blot analysis for caspase-9 and -3 was performed as described in materials and methods. The active, large subunits of caspase-9 were detected at ˜35 kDa (autocatalytic proteolysis) and ˜37 kDa (caspase-9 cleavage by caspase-3), the large subunits of active caspase-3 migrate at ˜17 kDa and 20 kDa. The small subunits (˜p10) of both caspases are not detected by our antibodies. (C) Capase-3 reconstitution in RL-7 cell lysates. Recombinant pro-caspase-3 (10 ng) was added to cytosolic extracts from RL-7 cells, activated with caspase-8 or cytochrome c/dATP and caspase activity was measured as described above.

FIG. 6. (A) K422 specificity exemplified by Brefeldin A and the staurosporine analog Staur-F3 (respective structures are show on the right). DOHH2 (▪), Jurkat (●), Jurkat bcl-2 (+), K422 (▴) and RL-7 (□) cells were treated with the indicated compound concentrations for 24 hours and cell viability was determined by MTT assays (data are the mean of triplicates). Brefeldin A and Staur-F3 induced caspase activation measured by enzymatic activity assays (AFC release from DEVD-AFC) (B) and Western blot analysis (C) and (D). After treatment with 100 nM Brefeldin A or 100 nM Staur-F3 for the indicated periods of time (in hours) total cell lysates were prepared and normalized for protein concentration. Caspase activity assays (10 μg total protein) and Western blot analysis (40 μg) for caspase-2, -3, -7, -8 and -9 were performed as described in materials and methods. The expression of β-actin was used to control equal protein loading and each experiment was repeated twice with similar results.

FIG. 7. (A) Correlation of course of disease and molecular events in FL.

FIG. 8. Model of “classic” and “transformation/survival specific” chemotherapy induced cell-death pathways.

### DEFINITIONS

A “cancer” in an animal refers to the presence of a physiological condition typified by unregulated cell growth. Cancer cells are generally characterized by features such as a rapid growth and proliferation rate, immortality, metastatic potential, and certain characteristic morphological features. Often, cancer cells will be in the form of a tumor, but such cells may exist alone within an animal, or may be present in bone marrow or circulate in the blood stream as independent cells, such as leukemic cells. A “cancer cell”, as used herein, is originally isolated from a cancer in an animal, e.g., a human, although the cell may also be passaged in cell culture and additionally manipulated, e.g., engineered to express particular proteins. The terms “cancer cells” and “tumor cells” are used interchangeably.

A “normal” cell as used herein is one that is not a cancer cell, i.e., does not exhibit hallmarks of cancer cells such as growth in soft agar, uncontrolled proliferation, and the like.

The term “chemoresistant tumor” or “chemoresistant cancer” as used herein refers to a cancer that does not undergo cell death in response to a chemotherapeutic agent, e.g., an agent that causes DNA damage.

The phrase “changes in cell growth” refers to any change in cell growth and proliferation characteristics in vitro or in vivo. Often, changes in cell growth with respect to the screening methods of the invention refer to changes in cell viability. However, “changes in cell growth” can include other characteristics such as anchorage independence, semi-solid or soft agar growth, changes in contact inhibition and density limitation of growth, changes in growth factor or serum requirements, changes in cell morphology, gaining or losing immortalization, changes in tumor-specific markers, ability to form or suppress tumors when injected into suitable animal hosts, and/or immortalization of the cell. See, e.g., Freshney, Culture of Animal Cells a Manual of Basic Technique pp. 231-241 (3rd ed. 1994).

The term “apoptosis” refers to the controlled form of cell death that is typically accompanied by one or more characteristic cell changes including condensation of cytoplasm, loss of plasma membrane microvilli, segmentation of the nucleus, degradation of chromosomal DNA, or loss of mitochondrial function. Apoptosis can be assessed using assays known to those in the art such as cell viability assays, FACS analysis, DNA electrophoresis, and the like.

The term “TRAIL receptor,” refers to a member of the tumor necrosis family (TNF) family of receptors that specifically binds to tumor necrosis factor related apoptosis inducing ligand (TRAIL) and mediates apoptosis. TRAIL receptors include DR5 and DR4.

A “defect in the TRAIL-DR5 pathway” refers to dysfunction of the extrinsic cell death pathway such that apoptosis is inhibited.

The term “antibody agonist” refers to an antibody capable of activating a receptor to induce a full or partial receptor-mediated response. For example, an agonist of DR5 binds to DR5 and induces DR5-mediated signaling. In some embodiments, a DR5 antibody agonist can be identified by its ability to bind to DR5 and induce apoptosis. Cells in which DR5 is know to be active in inducing apoptosis, e.g., Jurkat's cells, may be used to test agonist activity.

The term “apoptosis-inducing agent” refers to a compound that induces or promotes apoptosis in at least one cell type when contacted to the cell type. Exemplary apoptosis-inducing agents include, e.g., agonists or mimetics of the following: SMAC, Bax, Bik, Bok, Bim, Bak, Bid, Noxa, Puma, Hrk, or Bad; BH3, p53, TRAIL ligand, Fadd, Myc, and Mekk1, as well as antagonists or inhibitors of the following: 26S Proteasome inhibitors, c-flip, NFκB pathway, IAP family members (e.g., XIAP, cIAP1, cIAP2, NAIP, MLIAP/Livin, survivin), proteasome pathway members (e.g., E1, E2 and E3); kinases P13, Akt1, 2, and 3, Rip, Nik; CD40; Bcl2 family members (e.g., Bcl2, Bcl-xl, A1, Mcl1), osteoprotegrin. Additional exemplary apoptosis-inducing agents include, e.g., agents that enhance DR5 and DR4 expression and/or stability, agents that enhance caspase activity or stability, and agents that induce or enhance a DNA damage response. Agonist or mimetics in the above list include the gene products themselves, e.g., p53 is a p53 agonist. Antagonists include agents that directly inhibit activity and agents that indirectly inhibit activity through decreasing expression or stability of target molecule mRNA or protein.

An agent that “prevents or reduces” the growth of cancer cells refers to a compound that partially or totally blocks proliferation. Cell growth may be reduced by at least, e.g., 5%, 10%, 25%, 50%, 75%, 90%, 95% or 100% in comparison to control cells that are not treated with the agent. Typically, in the invention described herein, inhibition of cell growth results from cell death that is induced by the agent.

“Cytotoxic” or “cytotoxicity” as used herein refers to the ability to kill cells. Thus, a “cytotoxic” agent induces cell death. The term includes both apoptotic and necrotic cell death.

The term “selectively inhibitory” or “selectively cytotoxic” as used herein refers to a preferential effect of a compound on one population of cells vs. a population of cells to which it is being compared. A “selective” compound, e.g., a compound that increases the level of cell death in one population of cells compared to another, may increase cell death by at least, e.g., 5%, 10%, 25%, 50%, 75%, 90%, 95%, or 100% in comparison to its effects on the comparison population. Similarly, a “selective” compound that, e.g., inhibits growth of a cancer cell population compared to another population, may reduce growth by at least 5%, 10%, 25%, 50%, 75%, 90%, 95%, or 100% in comparison to its effects on the comparison population.

The term “target compound” refers to the compound or a group of compounds to be screened for biological activity or other properties, either on the solid support or after it has been removed from the solid support. The term “target compound” is used interchanageably herein with the term “small molecule.”

The term “chemical library” or “array” refers to an intentionally created collection of differing target compounds or molecules that can be prepared either synthetically or biosynthetically and that can be screened for biological activity in a variety of different formats (e.g., libraries of soluble compounds, libraries of compounds tethered to solid supports, etc.). The term is also intended to refer to an intentionally created collection of stereoisomers. The library comprises at least 2 members, preferably at least 10 members, more preferably at least 102 members and still more preferably at least 103 members. Particularly preferred libraries comprise at least 104 members, more preferably 105 members and still more preferably at least 106 members.

The term “antibody” refers to a polypeptide encoded by an immunoglobulin gene or functional fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

An exemplary immunoglobulin (antibody) structural unit comprises a tetramer. Each tetramer is composed of two identical pairs of polypeptide chains, each pair having one “light” (about 25 kDa) and one “heavy” chain (about 50-70 kDa). The N-terminus of each chain defines a variable region of about 100 to 110 or more amino acids primarily responsible for antigen recognition. The terms variable light chain (V_{L}) and variable heavy chain (V_{H}) refer to these light and heavy chains respectively.

Examples of antibody functional fragments include, but are not limited to, complete antibody molecules, antibody fragments, such as Fv, single chain Fv (scFv), disulfide-stabilized Fv (dsFv), protein fragments comprising complementarity determining regions (CDRs), V_{L} (light chain variable region), V_{H} (heavy chain variable region), Fab, F(ab)2′ and any combination of those or any other functional portion of an immunoglobulin peptide capable of binding to target antigen (see, e.g., Fundamental Immunology (Paul ed., 3d ed. 1993). As appreciated by one of skill in the art, various antibody fragments can be obtained by a variety of methods, for example, digestion of an intact antibody with an enzyme, such as pepsin; or de novo synthesis. Antibody fragments are often synthesized de novo either chemically or by using recombinant DNA methodology. Thus, the term antibody, as used herein, includes antibody fragments either produced by the modification of whole antibodies, or those synthesized de novo using recombinant DNA methodologies (e.g., single chain Fv) or those identified using phage display libraries (see, e.g., McCafferty et al., Nature 348:552-554 (1990)). The term antibody also includes bivalent or bispecific molecules, diabodies, triabodies, and tetrabodies. Bivalent and bispecific molecules are described in, e.g., Kostelny et al. (1992) J Immunol 148:1547, Pack and Pluckthun (1992) Biochemistry 31:1579, Hollinger et al., 1993, supra, Gruber et al. (1994) J Iminunol:5368, Zhu et al. (1997) Protein Sci 6:781, Hu et al. (1996) Cancer Res. 56:3055, Adams et al. (1993) Cancer Res. 53:4026, and McCartney, et al. (1995) Protein Eng. 8:301.

For preparation of monoclonal or polyclonal antibodies, any technique known in the art can be used (see, e.g., Kohler & Milstein, Nature 256:495-497 (1975); Kozbor et al., Immunology Today 4:72 (1983); Cole et al., pp. 77-96 in Monoclonal Antibodies and Cancer Therapy (1985)). “Monoclonal” antibodies refer to antibodies derived from a single clone. Techniques for the production of single chain antibodies (U.S. Pat. No. 4,946,778) can be adapted to produce antibodies to polypeptides of this invention. Also, transgenic mice, or other organisms such as other mammals, may be used to express humanized antibodies. Alternatively, phage display technology can be used to identify antibodies and heteromeric Fab fragments that specifically bind to selected antigens (see, e.g., McCafferty et al., Nature 348:552-554 (1990); Marks et al., Biotechnology 10:779-783 (1992)).

A “chimeric antibody” is an antibody molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity.

A “humanized” antibody is an antibody that retains the reactivity of a non-human antibody while being less immunogenic in humans. This can be achieved, for instance, by retaining the non-human CDR regions and replacing the remaining parts of the antibody with their human counterparts. See, e.g., Morrison et al., Proc. Natl. Acad. Sci. USA, 81:6851-6855 (1984); Morrison and Oi, Adv. Immunol., 44:65-92 (1988); Verhoeyen et al., Science, 239:1534-1536 (1988); Padlan, Molec. Immun., 28:489-498 (1991); Padlan, Molec. Immun., 31(3):169-217 (1994).

The phrase “specifically (or selectively) binds” to an antibody or “specifically (or selectively) immunoreactive with,” when referring to a protein or peptide, refers to a binding reaction that is determinative of the presence of the protein in a heterogeneous population of proteins and other biologics. Thus, under designated immunoassay conditions, the specified antibodies bind to a particular protein at least two times the background and do not substantially bind in a significant amount to other proteins present in the sample. Specific binding to an antibody under such conditions may require an antibody that is selected for its specificity for a particular protein. This selection may be achieved by subtracting out antibodies that cross-react with, e.g., DR5 molecules from other species. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Antibodies, A Laboratory Manual (1988), for a description of immunoassay formats and conditions that can be used to determine specific immunoreactivity). Typically a specific or selective reaction will be at least twice background signal or noise and more typically more than 10 to 100 times background.

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymers. As used herein, the terms encompass amino acid chains of any length, including full-length proteins, wherein the amino acid residues are linked by covalent peptide bonds.

“siRNA” refers to small interfering RNAs, that can cause post-transcriptional silencing of specific genes in cells, for example, mammalian cells (including human cells) and in the body, for example, mammalian bodies (including humans). The phenomenon of RNA interference is described and discussed in Bass, Nature 411: 428-29 (2001); Elbahir et al., Nature 411: 494-98 (2001); and Fire et al., Nature 391: 806-11 (1998); and WO 01/75164, where methods of making interfering RNA also are discussed. The siRNAs based upon the sequences and nucleic acids encoding the gene products disclosed herein typically have fewer than 100 base pairs and can be, e.g., about 30 bps or shorter, and can be made by approaches known in the art, including the use of complementary DNA strands or synthetic approaches. Exemplary siRNAs according to the invention can have up to 29 bps, 25 bps, 22 bps, 21 bps, 20 bps, 15 bps, 10 bps, 5 bps or any integer thereabout or therebetween. Tools for designing optimal inhibitory siRNAs include that available from DNAengine Inc. (Seattle, Wash.) and Ambion, Inc. (Austin, Tex.).

### DETAILED DESCRIPTION OF THE INVENTION

This invention provides methods of identifying agents that can be used to inhibit the growth of chemoresistant cancers. Further, the invention provides methods of cancer diagnosis and prognosis based on the identification of mutations in the TRAIL-DR5 apoptotic pathway in chemoresistant cancer cells.

Identification of Agents that are Selectively Cytotoxic to Chemoresistant Cancer Cells

In one aspect, this invention provides a screening strategy to identify compounds that selectively target a chemoresistant cancer cell population. Agents that preferentially inhibit the growth of chemosresistant, but not normal cells, e.g., by inducing cell death, may be identified using the methods disclosed herein. Further, agents that selectively inhibit the growth of a chemoresistant cancer population having a particular defect in the TRAIL-DR5 pathway, e.g., a p53 mutation that reduces apoptosis, may also be identified using the methods of the invention. Compounds identified in the screening assays may be used as tools to probe alternative cell-death pathways, identify cell-death regulatory molecules, or used to treat cancer.

The screening methods of the invention employ tumor cells that are resistant to one or more chemotherapeutic agents and/or classes of chemotherapeutic agents. Anticancer drugs are traditionally classified as alkylating agents; antimetabolites, mitotic inhibitors, nucleotide analogs or inhibitors of topoisomerases. Many of these function primarily through DNA damage. Alkylating agents include mechoethamite, chlorambucil, melphalan, cyclophosphamide, ifosfamide, thiotep and busulfan; Other alkylating agents act through more complex mechanisms; these include dacarbazine, carnustine, lomustine, cisplatin, carboplatin, procarbazine and altretamine. Agents from natural produces can also cause DNA damage. For example, antibiotics can bind to DNA with subsequent distortion of the structure, and may also damage DNA by the formation of free radicals and chelation of metal ions. Such compounds derived from natural sources include doxorubicin, daunorubicin, idarubicin, mitoxantrone, bleomycin, dactinomycin, mitomycin C, plicamycin and streptozocin. Topoisomerase inhibitors, e.g, epipodophyllotoxins such as eptoposide, also induce DNA strand breakage. The chemoresistant cancer cells useful to the practitioner are typically refractory to treatment with chemotherapeutic agents for which the primary mechanism of action is through DNA damage. As noted above, these agents include alkylating agents, antibiotics, and topoisomerase inhibitors. Chemoresistant tumor cells are typically insensitive to more than one of these DNA damaging agents.

Chemoresistant tumor cells used in the invention have one or more mutations that prevent activation of the extrinsic, intrinsic, or both, apoptotic pathways. Defects may occur in the function of any of these members of the pathways (for review of extrinsic and intrinsic pathways, see, e.g., Igney & Krammer, NATURE REVIEWS CANCER 2:277-288, 2002, and references cited therein). Exemplary proteins that may be defective (e.g., mutated or changed in levels of expression) in chemoresistant tumors include, e.g., DR4, DR5, p53, anti-apoptotoic Bcl-2 family members (e.g., Bcl-2, Bcl-10, Bcl-xl, A1, Mcl1); pro-apoptotic Bcl-2 family members, (e.g., Bax, Bcl-xs, Bid, Bad, Bik, Bok, Bim, Bak, Noxa, Puma, or Hrk); IAP family members (e.g., XIAP, cIAP1, cIAP2, NAIP, MLIAP/Livin, survivin), death domain proteins, e.g., Fadd, TRADD; and caspases, e.g., caspase 8, caspase 7, caspase 9, caspase 3; as well as Myc; Mekk1; c-flip, NFκB pathway members; proteasome pathway members (e.g., E1, E2 and E3); kinases, P13, Akt1, 2, and 3, Rip, Nik;CD40; fas, and TNF receptor. Often, the cells have a defect in the TRAIL-DR5 pathway. Further, the chemoresistant tumors often have a defect in a bcl-2 family member, e.g, they may overexpress bcl-2, which can repress the mitochondria-dependent pathway. Other common mutations include those that inactivate or reduce the activity of p53, e.g., mutations in the gene encoding p53, which can participate in both the intrinsic and extrinsic pathways, and thereby affecting both the intrinsic and extrinsic cell death pathways. Similarly, caspase-3 mutations may affect both cell death pathways, whereas mutations in caspase-9 or caspase-8 may preferentially inhibit one of the pathways.

In some embodiments, at least two populations of tumor cells that differ in at least one defect in a cell death pathway are screened for sensitivity to the candidate agents. The screening assay comparing the two different chemoresistant populations may, but need not necessarily, include a step of screening normal cells for sensitivity to the agent. For example, two tumor cell populations, which may or may not be from the same patient, are used in the screen. Both overexpress bcl-2 and have defects in p53, but the mutations are different. These two populations are considered to be different populations.

Identification of Apoptosis Pathway Defects

Detection of Apoptosis-Associated Polynucleotides

Mutations in members of the intrinsic and extrinsic apoptotic pathways, e.g., those listed above, may be determined using a number of different methods that detect mutations or changes in the levels of nucleic acids encoding the proteins, or that detect dysfunctioning polypeptides or altered polypeptide levels. Often, chemoresistant cancer cells may optionally first be directly screened for reduced levels of apoptosis, for example, in response to application of an alkylating agent or a topoisomerase inhibitor, and/or response to a TRAIL receptor agonist, e.g., TRAIL or an apo-2 receptor ligand such as an antibody agonist. Assays for detecting apoptosis are further described below. Those cells that exhibit decreased cell death in comparison to normal cells or chemosensitive cancer cells may then further be analyzed to detect specific defects in the apoptotic pathways.

Detection of mutations may involve quantitative or qualitative detection of the polypeptide or polynucleotide. A polynucleotide level may be detected by determining the level of a DNA or RNA. A mutant polypeptide or changes in polypeptide levels may be detected by detecting the polypeptide, for example using an immunoassay, or by detecting protein activity, e.g., caspase activity.

In one embodiment, the presence of a dysfunctional apoptotic pathway member is evaluated by assessing nucleic acid sequences that encode apoptosis-associated proteins. Such an analysis can be performed, for example, using hybridization assays, amplification assays, sequencing assays, and combinations of these procedures. The methodology for these tehcniques is well known in the art and described, for example, in basic texts disclosing the general methods of use in this invention (see, e.g., Sambrook and Russell, Molecular Cloning, A Laboratory Manual (3rd ed. 2001); Current Protocols in Molecular Biology (Ausubel et al., eds., 1999); and Current Protocols in Human Genetics, (Dracopoli et al, eds, 2000)).

A variety of methods of specific DNA and RNA measurement that use nucleic acid hybridization techniques are known to those of skill in the art. Some methods involve an electrophoretic separation (e.g., Northern blot for detecting RNA), but measurement of DNA and RNA can also be carried out in the absence of electrophoretic separation (e.g., by dot blot, RNase protection assays, in situ hybridizations, various amplification reactions). The selection of a nucleic acid hybridization format is not critical. A variety of nucleic acid hybridization formats are known to those skilled in the art. Hybridization techniques are generally described in Hames and Higgins Nucleic Acid Hybridization, A Practical Approach, IRL Press (1985); Gall and Pardue, Proc. Natl. Acad. Sci. U.S.A., 63:378-383 (1969); and John et al. Nature, 223:582-587 (1969).

In one hybrization format, nucleic acid levels of multiple components of apoptotic pathways may conveniently be measured in an array format. In some embodiments, the target nucleic acid or the probe is immobilized on a solid support. Solid supports suitable for use in the assays of the invention are known to those of skill in the art. As used herein, a solid support is a matrix of material in a substantially fixed arrangement. A variety of automated solid-phase assay techniques are also appropriate. For instance, very large scale immobilized polymer arrays (VLSIPS™), i.e. Gene Chips or microarrays, available from Affymetrix, Inc. in Santa Clara, Calif. can be used to detect changes in expression levels of a plurality of genes involved in the same regulatory pathways simultaneously. See, Tijssen, supra., Fodor et al. (1991) Science, 251: 767-777; Sheldon et al. (1993) Clinical Chemistry 39(4): 718-719, and Kozal et al. (1996) Nature Medicine 2(7): 753-759. Similarly, spotted cDNA arrays (arrays of cDNA sequences bound to nylon, glass or another solid support) can also be used to monitor expression of a plurality of genes.

In another embodiment, amplification-based assays are used to measure the level of expression of apoptosis-related polynucleotides. In a quantitative amplification, the amount of amplification product will be proportional to the amount of template in the original sample. Methods of quantitative amplification are well known to those of skill in the art. Detailed protocols for quantitative PCR are provided, e.g., in Innis et al. (1990) PCR Protocols, A Guide to Methods and Applications, Academic Press, Inc. N.Y.). For example, a quantitative RT-PCR reaction such as a TaqMan based assay may be used to quantify the apoptosis-associated polynucleotides. Other suitable amplification methods include, but are not limited to, ligase chain reaction (LCR) (see, Wu and Wallace (1989) Genomics 4: 560, Landegren et al. (1988) Science 241: 1077, and Barringer et al. (1990) Gene 89: 117), transcription amplification (Kwoh et al. (1989) Proc. Natl. Acad. Sci. USA 86: 1173), self-sustained sequence replication (Guatelli et al. (1990) Proc. Nat. Acad. Sci. USA 87: 1874), dot PCR, and linker adapter PCR, etc.

Specific mutations may also be determined by sequencing the nucleic acids, for example, those encoding p53, to determine if there are mutations present relative to the wildtype sequence. Sequencing is commonly performed in conjunction with amplification techniques to obtain adequate amounts of nucleic acid.

Detection of Apoptosis-Associated Polypeptides

Defects in apoptotic pathways may also be identified by measuring the levels of proteins in the cancer cells. For example, immunoassays can be used to qualitatively or quantitatively analyze polypeptides. A general overview of the applicable technology can be found in Harlow & Lane, Antibodies: A Laboratory Manual (1988) and Harlow & Lane, Using Antibodies: A Laboratory Manual (1999).

Antibodies specific for various apoptosis-associated proteins are known in the art. For example, anti-DR5 antibodies are available commercially (e.g., from Cayman Chemicals) as are various other antibodies, e.g, Fas antibodies (MBL), p53 antibodies (Pharmingen), caspase 3 antibodies (Pharmingen), caspase 8 antibodies (Pharmingen), caspase 9 antibodies (Pharmingen), and bcl-2 antibodies (Pharmingen).

Antibodies that selectively bind apoptosis-specific proteins may also be produced. Methods of producing specific polyclonal and monoclonal antibodies are known to those of skill in the art (see, e.g., Coligan, Current Protocols in Immunology (1991); Harlow & Lane, supra; Goding, Monoclonal Antibodies: Principles and Practice (2d ed. 1986); and Kohler & Milstein, Nature 256:495-497 (1975)). These techniques include antibody preparation by selection of antibodies from libraries of recombinant antibodies in phage or similar vectors, as well as preparation of polyclonal and monoclonal antibodies by immunizing rabbits or mice. Such antibodies can be used for analysis of cancer cells for use in candidate compound screening protocols as well as for diagnostic/prognostic and therapeutic applications.

Apoptosis-associated proteins can be detected and/or quantified using any of a number of well recognized immunological binding assays. For a review of the general immunoassays, see Harlow & Lane, supra; and also Methods in Cell Biology: Antibodies in Cell Biology, volume 37 (Asai, ed. 1993); and Basic and Clinical Immunology (Stites & Terr, eds., 7th ed. 1991). Immunoassays may be in a competitive or non-competitive format. Often, an ELISA or western blot is used to determine levels of expression of polypeptides.

Alternatively, dysfunction of particular proteins in the apoptotic pathway may be assessed by analyzing activity, e.g, capase activity. Caspase activities can be assessed using known techniques such as those outlined in Example 1, which measure the protease activity. Assays that measure activity of other apoptotic pathway members, e.g., various kinases, are also performed using known methods, such as phosphorylation assays.

Inhibition of Cancer Cell Growth

The compound screening assays of the invention identify agents that can selectively inhibit the growth of chemoresistant tumor cells, e.g., by inducing cell death via either apoptosis or necrosis. Inhibition of growth may be measured using a number of common assays. Such assays may measure cell viability or cell number, either directly or indirectly. Many such assays and assay systems are known in the art and commercially available. These include those that generate signals via fluorescence, calorimetrically, by radioactivity, or other common signaling systems.

In some embodiments, cell viability in response to a candidate compound is tested in a chemoresistant cancer cell population and a normal cell population (and/or a second chemoresistant cancer cell population). Cell viability may be assessed by measuring many different endpoints including levels of cytoplasmic enzymes, permeability of cells to dyes, DNA fragmentation, release of a radioisotopic label such as ⁵¹Cr or other formats. Typically, cell viability is measured using an assay suitable for a high throughput screening format, such as a colorimetric or fluorescent viability assay. For example, an Alamar blue (AB) assay, incorporates a redox indicator that changes the colour or fluorescence in response to metabolic activity. The Alamar blue fluoresces in the presence of living, but not dead, cells. Such an assay can be conviently read in a microplate or by flow cytometry. Colorimetric assays such as the MTT assay, which measures the reduction of MTT (3-(4.5-dimethyl) thiazol-2-yl-2,5-diphenyl tetrazolium bromide) to formazan, may also be used conveniently in a high throughput format to measure cell viability and proliferation.

Other assays that measure cell number may also be used. These include assays that measure intercalation of dyes into the DNA of a cell. The amount of intercalated dye is directly proportional to cell number. For example, cells can be stained with a dye such as Hoechst 33342, which intercalates in the DNA of vital cell, an cell number determined by measuring the amount of fluorescence. Cells may also be directly counted.

Candidate Compounds and High Throughput Screening

The method of the invention also provide compounds that exhibit selective cell growth inhibitory effects, e.g., selective cytotoxicity, on chemoresistant tumor populations. The compounds tested for selectivity toward particular populations of tumor cells can be any small chemical compound, or a biological entity, e.g., a macromolecule such as a protein, sugar, nucleic acid or lipid. Thus, test compounds may be small chemical molecules; combinatorial chemical libraries; nucleic acids, including oligonucleotides, anti-sense oligonucleotides, siRNAs, etc., polypeptides, including antibodies, antibody fragments, and short peptides; extracts, e.g,, from natural sources; and the like.

Typically, test compounds will be small chemical molecules and polypeptides. The assays of the invention are typically designed to screen large chemical libraries by automating the assay steps, which are typically run in parallel (e.g., in microtiter formats on microtiter plates in robotic assays). It will be appreciated that there are many suppliers of chemical compounds, including Sigma (St. Louis, Mo.), Aldrich (St. Louis, Mo.), Sigma-Aldrich (St. Louis, Mo.), Fluka Chemika-Biochemica Analytika (Buchs Switzerland) and the like.

In one preferred embodiment, high throughput screening methods are employed. These methods involve providing a combinatorial chemical or peptide library containing a large number of potential therapeutic compounds. Such “combinatorial chemical libraries” are then screened in one or more assays, such as cell viability assays as described herein, to identify those library members (particular chemical species or subclasses) that display the desired characteristic activity, e.g., selective cytotoxocity to chemoresistant tumor cells. The compounds thus identified can serve as conventional “lead compounds” or can themselves be used as potential or actual therapeutics.

A combinatorial chemical library is a collection of diverse chemical compounds generated by either chemical synthesis or biological synthesis, by combining a number of chemical “building blocks” such as reagents. For example, a linear combinatorial chemical library such as a polypeptide library is formed by combining a set of chemical building blocks (amino acids) in every possible way for a given compound length (i.e., the number of amino acids in a polypeptide compound). Millions of chemical compounds can be synthesized through such combinatorial mixing of chemical building blocks.

Preparation and screening of combinatorial chemical libraries is well known to those of skill in the art. Such combinatorial chemical libraries include, but are not limited to, peptide libraries (see, e.g., U.S. Pat. No. 5,010,175, Furka, Int. J. Pept. Prot. Res. 37:487-493 (1991) and Houghton et al., Nature 354:84-88 (1991)). Other chemistries for generating chemical diversity libraries can also be used. Such chemistries include, but are not limited to: peptoids (e.g., PCT Publication No. WO 91/19735), encoded peptides (e.g., PCT Publication WO 93/20242), random bio-oligomers (e.g., PCT Publication No. WO 92/00091), benzodiazepines (e.g., U.S. Pat. No. 5,288,514), diversomers such as hydantoins, benzodiazepines and dipeptides (Hobbs et al., Proc. Nat. Acad. Sci. USA 90:6909-6913 (1993)), vinylogous polypeptides (Hagihara et al., J. Amer. Chem. Soc. 114:6568 (1992)), nonpeptidal peptidomimetics with glucose scaffolding (Hirschmann et al., J. Amer. Chem. Soc. 114:9217-9218 (1992)), analogous organic syntheses of small compound libraries (Chen et al., J. Amer. Chem. Soc. 116:2661 (1994)), oligocarbamates (Cho et al., Science 261:1303 (1993)), and/or peptidyl phosphonates (Campbell et al., J. Org. Chem. 59:658 (1994)), nucleic acid libraries (see Ausubel, Berger and Russell & Sambrook, all supra), peptide nucleic acid libraries (see, e.g., U.S. Pat. No. 5,539,083), antibody libraries (see, e.g., Vaughn et al., Nature Biotechnology, 14(3):309-314 (1996) and PCT/US96/10287), carbohydrate libraries (see, e.g., Liang et al., Science, 274:1520-1522 (1996) and U.S. Pat. No. 5,593,853), small organic molecule libraries (see, e.g., benzodiazepines, Baum C&EN, Jan 18, page 33 (1993); isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337; benzodiazepines, 5,288,514, and the like).

Devices for the preparation of combinatorial libraries are commercially available (see, e.g., 357 MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.). In addition, numerous combinatorial libraries are themselves commercially available (see, e.g., ComGenex, Princeton, N.J., Tripos, Inc., St. Louis, Mo., 3D Pharmaceuticals, Exton, Pa., Martek Biosciences, Columbia, Md., etc.).

Candidate compounds include numerous chemical classes; however, typically they are small organic molecules, generally having a molecular weight of more than about 100 and less than about 2, 500 daltons. Typical small molecules are less than about 2000, less than about 1500, less than about 1000, or less than about 500 daltons. The candidate compounds comprise functional groups necessary for structural interactions with proteins, e.g., hydrogen bonding, and typically include at least an amine, carbonhyl, hydroxyl, or carboxyl group, preferably at least two of the functional chemical groups. The candidate agents often comprise cyclical carbon or heterocyclic structures and/or aromatic or polyaromatic structures substituted with one or more of the above functional groups. candidate compounds include peptides, saccharides, fatty acids, steroids, purines, pyrimidines, and various structural analogs or combinations thereof.

High Throughput Assays

In the high throughput assays of the invention, it is possible to screen thousands of different modulators in a single day. In particular, each well of a microtiter plate, e.g., a 96, 384, or 1,536-well plate, can be used to run a separate assay against a selected potential modulator, or, if concentration or incubation time effects are to be observed, every 5-10 wells can test a single modulator. Thus, a single standard microtiter plate can assay a large number of modulators. For example, if 1536-well plates are used, then a single plate can easily assay from about 100-1500 different compounds. It is possible to assay many different plates, for example over 1 million wells per days, using high throughput systems, for example those described in WO02/31747. Thus, many thousands of compounds can be screened in a single day.

High throughput systems comprise automated components, including fluid transfer and dispensing devices. A number of fluid transfer systems are available, or can easily be made from existing components. For example, a Zymate XP (Zymark Corporation; Hopkinton, Mass.) automated robot using a Microlab 2200 (Hamilton; Reno, Nev.) pipetting station can be used to transfer parallel samples to microtiter plates to set up several parallel simultaneous assays. Such a fluid transfer device typically comprises an array of receptacles arranged such that the outlets of the receptacle are aligned with wells on the microwell plate. The Robbins Hydra (Robbins, Scientific, Sunnyvale, Calif.) is another example of a fluid dispensing device that can also be used in high throughput screening systems. Other fluid manipulation devices may include those that incorporate positive displacement pumps and dispenser valves, such a Cartesian SynQUAD (U.S. Pat. No. 6,063,339, available from Cartesian Technologies, Inc., Irvine, Calif.).

As appreciated by one of skill in the art, the high throughput devices used in the screening methods may also comprise additional components such as an incubator, e.g., to provides particular growth conditions for cells.

Detectors may also be included in the high throughput assay system. The detectors may measure any physical property of a sample. For example, fluorescence, luminescence, phosphorescence, radioactivity, or any other physical property may be measured by the detector. Examples of detectors that are often used in cell-based high throughput screening assays include a Fluormetric Imaging Plate Reader System (FLIPR®), which is commercially available from Molecular Devices Corp. Sunnyvale, Calif.; and a chemiluminescnet imaging plate reader (CLIPR™). Additional imaging systems are described, e.g., in WO00/17643.

Optical images viewed (and, optionally, recorded) by a camera or other recording device (e.g., a photodiode and data storage device) are optionally further processed in any of the embodiments herein, e.g., by digitizing the image and storing and analyzing the image on a computer. A variety of commercially available peripheral equipment and software is available for digitizing, storing and analyzing a digitized video or digitized optical image for high throughput systems.

One conventional system carries light from the specimen field to a cooled charge-coupled device (CCD) camera, in common use in the art. A CCD camera includes an array of picture elements (pixels). The light from the specimen is imaged on the CCD. Particular pixels corresponding to regions of the specimen (e.g., individual hybridization sites on an array of biological polymers) are sampled to obtain light intensity readings for each position. Multiple pixels are processed in parallel to increase speed. This type of apparatus is easily used for viewing any sample, e.g., by fluorescent or dark field microscopic techniques.

Administration and Pharmaceutical Compositions

Selectively inhibitory compounds can be administered to a mammalian subject to inhibit growth of cancer cells. As described in detail below, the inhibitory compounds are administered in any suitable manner, optionally with pharmaceutically acceptable carriers. These compounds may be used in conjunction with other therapeutic treatments. The compositions are administered to a patient in an amount sufficient to elicit an effective protective or therapeutic response in the patient. An amount adequate to accomplish this is defined as “therapeutically effective dose.” The dose will be determined by the efficacy of the particular inhibitors employed and the condition of the subject, as well as the body weight or surface area of the area to be treated. The size of the dose also will be determined by the existence, nature, and extent of any adverse effects that accompany the administration of a particular compound or vector in a particular subject.

Toxicity and therapeutic efficacy of such compounds can be determined by standard pharmaceutical procedures in cell cultures or experimental animals, for example, by determining the LD₅₀ (the dose lethal to 50% of the population) and the ED₅₀ (the dose therapeutically effective in 50% of the population). The dose ratio between toxic and therapeutic effects is the therapeutic index and can be expressed as the ratio, LD₅₀/ED₅₀. Compounds that exhibit large therapeutic indices are preferred. While compounds that exhibit toxic side effects can be used, care should be taken to design a delivery system that targets such compounds to the site of affected tissue to minimize potential damage to normal cells and, thereby, reduce side effects.

The data obtained from cell culture assays and animal studies can be used to formulate a dosage range for use in humans. The dosage of such compounds lies preferably within a range of circulating concentrations that include the ED₅₀ with little or no toxicity. The dosage can vary within this range depending upon the dosage form employed and the route of administration. For any compound used in the methods of the invention, the therapeutically effective dose can be estimated initially from cell culture assays. A dose can be formulated in animal models to achieve a circulating plasma concentration range that includes the IC₅₀ (the concentration of the test compound that achieves a half-maximal inhibition of symptoms) as determined in cell culture. Such information can be used to more accurately determine useful doses in humans. Levels in plasma can be measured, for example, by high performance liquid chromatography (HPLC). In general, the dose equivalent of a modulator is from about 1 ng/kg to 10 mg/kg for a typical subject.

Pharmaceutical compositions for use in the present invention can be formulated by standard techniques using one or more physiologically acceptable carriers or excipients. The compounds and their physiologically acceptable salts and solvates can be formulated for administration by any suitable route, including via inhalation, topically, nasally, orally, parenterally (e.g., intravenously, intraperitoneally, intravesically or intrathecally) or rectally.

For oral administration, the pharmaceutical compositions can take the form of, for example, tablets or capsules prepared by conventional means with pharmaceutically acceptable excipients, including binding agents, for example, pregelatinised maize starch, polyvinylpyrrolidone, or hydroxypropyl methylcellulose; fillers, for example, lactose, microcrystalline cellulose, or calcium hydrogen phosphate; lubricants, for example, magnesium stearate, talc, or silica; disintegrants, for example, potato starch or sodium starch glycolate; or wetting agents, for example, sodium lauryl sulphate. Tablets can be coated by methods well known in the art. Liquid preparations for oral administration can take the form of, for example, solutions, syrups, or suspensions, or they can be presented as a dry product for constitution with water or other suitable vehicle before use. Such liquid preparations can be prepared by conventional means with pharmaceutically acceptable additives, for example, suspending agents, for example, sorbitol syrup, cellulose derivatives, or hydrogenated edible fats; emulsifying agents, for example, lecithin or acacia; non-aqueous vehicles, for example, almond oil, oily esters, ethyl alcohol, or fractionated vegetable oils; and preservatives, for example, methyl or propyl-p-hydroxybenzoates or sorbic acid. The preparations can also contain buffer salts, flavoring, coloring, and/or sweetening agents as appropriate. If desired, preparations for oral administration can be suitably formulated to give controlled release of the active compound.

For administration by inhalation, the compounds may be conveniently delivered in the form of an aerosol spray presentation from pressurized packs or a nebulizer, with the use of a suitable propellant, for example, dichlorodifluoromethane, trichlorofluoromethane, dichlorotetrafluoroethane, carbon dioxide, or other suitable gas. In the case of a pressurized aerosol, the dosage unit can be determined by providing a valve to deliver a metered amount. Capsules and cartridges of, for example, gelatin for use in an inhaler or insufflator can be formulated containing a powder mix of the compound and a suitable powder base, for example, lactose or starch.

The compounds can be formulated for parenteral administration by injection, for example, by bolus injection or continuous infusion. Formulations for injection can be presented in unit dosage form, for example, in ampoules or in multi-dose containers, with an added preservative. The compositions can take such forms as suspensions, solutions, or emulsions in oily or aqueous vehicles, and can contain formulatory agents, for example, suspending, stabilizing, and/or dispersing agents. Alternatively, the active ingredient can be in powder form for constitution with a suitable vehicle, for example, sterile pyrogen-free water, before use.

The compounds can also be formulated in rectal compositions, for example, suppositories or retention enemas, for example, containing conventional suppository bases, for example, cocoa butter or other glycerides.

Furthermore, the compounds can be formulated as a depot preparation. Such long-acting formulations can be administered by implantation (for example, subcutaneously or intramuscularly) or by intramuscular injection. Thus, for example, the compounds can be formulated with suitable polymeric or hydrophobic materials (for example as an emulsion in an acceptable oil) or ion exchange resins, or as sparingly soluble derivatives, for example, as a sparingly soluble salt.

The compositions can, if desired, be presented in a pack or dispenser device that can contain one or more unit dosage forms containing the active ingredient. The pack can, for example, comprise metal or plastic foil, for example, a blister pack. The pack or dispenser device can be accompanied by instructions for administration.

Diagnostic/Prognostic Applications

According to the present invention, a “method of inhibiting the growth of cancer cells” refers to a procedure or course of action that is designed to reduce or eliminate the number of cancer cells in an animal, or to alleviate the symptoms of a cancer. Treating cancer or inhibiting the growth of cancer cells does not necessarily mean that the cancer cells will, in fact, be eliminated, that the number of cells will, in fact, be reduced, or that the symptoms of a cancer will, in fact, be alleviated. Often, a method of treating cancer will be performed even with a low likelihood of success, but which, given the medical history and estimated survival expectancy of an animal, is nevertheless deemed an overall beneficial course of action.

The detection of one or more mutations in an apoptotic pathway can be used to determine the diagnosis and/or prognosis of a mammal with cancer and aid in determining a preferred treatment. For example, in certain embodiments, the invention provides methods for treating cancer by detecting the level and/or a diagnostic presence of a defect in the DR5 TRAIL pathway in a biological sample, and, when a diagnostic presence or increased level is detected, applying a non-DNA damaging anti-cancer therapy or combination therapy with DNA-damaging anti-cancer agents.

Chemoresistant tumor cells typically have a dysfunction in one or more apoptotic pathways. Tumor cells may be analyzed prior to, or during treatment, to monitor the occurrence of such mutations in populations of cancer cells. Different mutations may occur in subpopulations of the cells present within a single patient. Based on the presence or absence of particular mutations, e.g., mutations in p53, a decision to add a therapeutic agent to the treatment protocol, e.g., a non-DNA damaging agent, or to discontinue administration of a particular therapy, e.g., an alkylating agent, may then be made. For example, detection of a p53 mutation that inhibits p53 in a tumor cell sample from a tumor that overexpresses bcl-2, e.g, a follicular lymphoma, may lead to a decision to add another treatment agent that does not induce DNA damage, for example, an agent such as an anti-CD20 antibody, an allogeneic T-lymphocyte, a bcl-2 inhibitor, or a reagent that activates the TRAIL receptor, e.g., a DR5 antibody agonist, or upregulates expression of the TRAIL receptor.

In certain embodiments, immunotherapy will be used to treat a cancer following a diagnosis based on detection of a defect in the DR5 TRAIL receptor pathway. Methods of enhancing the ability of an animal's immune system to destroy cancer cells within the animal are known in the art. Numerous such methods are well known to those of skill in the art. This can involve the treatment with polyclonal or monoclonal antibodies that bind to particular molecules located on, produced by, or indicative of, tumor cells, e.g., antibodies identified above. In some embodiments, an anti-DR4 or anti-DR5 antibody agonist may be administered to an individual undergoing treatment for cancer. Anti-DR5 antibodies have been described previously in, e.g., PCT WO 01/83560 (antibody TRA-8; ATCC PTA-1428) and PCT WO 02/079377. In other embodiments, e.g., the treatment of lymphomas, anti-CD20 antibodies, e.g., Rituximab, are administered alone or in conjunction with other agents. Immunotherapeutic methods involving administration of therapeutic antibodies, alone or conjugated to a toxin, are well know to those of skill in the art (see, e.g., Pastan et al.(1992) Ann. Rev. Biochem., 61: 331-354, Brinkman and Pastan (1994) Biochimica Biphysica Acta, 1198: 27-45, etc.). Other, non-antibody based, immunotherapies, e.g., administration of allogeniec lymphocytes (see, e.g., U.S. Pat. No. 5,843,435, U.S. Pat. No. 6,207,147 and references cited therein), may also be used alone or in comibantion with other therapies to treat chemoresistant cancers that have a defect in the TRAIL DR5 pathway.

Thus, the invention provides numerous methods for optimizing treatment of a mammal with a cancer that comprises a population of cells having one or more defects in apoptosis, in particular, in the TRAIL DR5 pathway. It will be appreciated that based on the status of the patient and other clinical factors, the non-DNA damaging compounds may also be administered in conjunction with one or more traditional, well known anti-cancer therapies, e.g., chemotherapy, radiation therapy, surgery, hormone therapy, immunotherapy, anti-angiogenic therapy etc.

### EXAMPLES

### Example 1

### Identification of Mechanisms Underlying Chemoresistance and Selection of Compounds that Inhibit Growth of Chemoresistant Lymphoma Cells

This example provides an illustration of screening for compounds that inhibit cancer growth using chemoresistant cancer cells.

Methods

Materials

Fluorogenic AFC caspase substrate (Ac-DEVD-AFC), and the caspase inhibitors Z-IETD-fmk and Z-LEHD-fmk were purchased from Calbiochem. Cell culture reagents were purchased from Gibco and all other chemicals (including etoposide and doxorubicin) from Sigma.

Lymphomas cells DOHH2, K422, RL-7 and Jurkat cells were cultured in RPMI1640 supplemented with 10% heat inactivated fetal bovine serum, 2 mM L-glutamine, 10 mM HEPES and 1.0 mM sodium pyruvate. DOHH2 and K422 cell lines were obtained from from DSMZ (Braunschweig, Germany), RL-7 cells were provided by Dr. Bertrand Nadel (University of Vienna, Austria), and Jurkat cells were purchased from ATCC.

An MTT cytotoxicity assay was used to determine the number of viable cells following treatment with various apoptotic stimuli. Cells (100 μl of a 8×10⁵ cells/ml suspension) were added to each well of a 96 well plate and incubated with etoposide or doxorubicin for 24-72 h. MTT dye solution was then added for 4 h, the blue crystal product was solubilized over night at 37° C. and absorbance was measured at 570 nm. Because the MTT assay also measures growth arrest, the MTT results were verified with caspase activity assays.

Western blot analysis was used to evaluate protein expression. Cells (2.4×10⁶ well) were seeded in 6 well plates and treated with the respective apoptosis stimuli. After the indicated time periods cells were washes in ice cold PBS and lysed in 1% Triton X-100 containing hypotonic lysis buffer (20 mM HEPES pH 7.5, 10 mM KCl, 1.5 mM MgCl₂, 1 mM EDTA and 1 mM DTT). Protein (40 μg) was electorphoresed under reducing conditions on 10-20% gradient SDS-polyacrylamide gels. After electroblotting on nitrocellulose membranes (Schleicher and Schuell) the membranes were blocked for 1 h with 10% non-fat milk powder and immunoblotted for 1 h with the following primary antibodies: rabbit anti-DR5 polyclonal antibody (1:500, Cayman Chemicals), mouse anti-Fas monoclonal antibody CH-11 (MBL), rabbit anti-caspase-3 polyclonal antibody (1:1000, Pharmingen), rabbit anti-caspase-8 polyclonal antibody (1:1000, Pharmingen), mouse anti-caspase-9 monoclonal antibody 5B4 (1:1000, MBL), mouse anti-bcl-2 monoclonal antibody (1:500, Pharmingen) or goat anti-actin polyclonal antibody (1:200, Santa Cruz Biotechnology). Membranes were washed five times with TBS/0.05% Tween, incubated with the respective peroxidase conjugated affinity purified secondary antibody (1:5000, Biorad) for 30 min, washed again five times and developed using enhanced chemiluminescence (ECL, Amersham).

Isolation of DR5 Antibody Agonists

DR5 antibody agonists were isolated using a functional screen. Briefly, Balb-C mice were immunized with a thioredoxin-DR5 fusion protein. Immunization was performed using the RIMMS (Repetitve Immunization at Multiple Sites) procedure. B-cells isolated from pooled peripheral lymph nodes were fused to bcl-2 transfected myeloma cells. Supernatants from the resulting hybridomas were cross-linked with goat anti-mouse Fc and assayed for functional activity in an Alamar Blue redox screen. Positive wells were identified, hybridomas were further sub-cloned by limiting dilution, and monoclonal antibody was isolated for further characterization. Subsequently, the antibodies were shown to be DR5, but not DR1, DR2, DR3 or DR4, specific and capable of activating caspase-3 in a variety of DR5 expressing tumor cell lines.

Caspase Assays

Caspase activities were assayed as previously described (Stennicke and Salvesen, 2000) at 37° C. in 40 μl of caspase buffer (50 mM HEPES pH 7.4, 100 mM NaCl, 10% sucrose, 1 mM EDTA, 0.1% CHAPS and 10 mM DTT) containing 100 μM of the fluorogenic peptide Ac-DEVD-AFC. Activity was measured continuously over the indicated time by the release of AFC from DEVD-AFC using a Molecular Devices fluorometer in the kinetic mode and with the 405-510 filter pair. For the assessment of caspase activity in intact cells after treatment with different apoptosis stimuli 10-20 μg of total cell protein (Triton X-100 extracts) was used in 40 μl of caspase buffer (containing 100 μM DEVD-AFC). For in-vitro initiation of caspase activity by cytochrome c Triton X-100 free, hypotonic cytosolic extracts were prepared and 10 μM horse heart cytochrome c (Sigma) together with 1 mM dATP or recombinant active caspase-8 (20 nM) were used for caspase activation (Deveraux et al., 1997). For reconstitution of RL-7 hypotonic cell extracts recombinant pro-caspase-3 was used as previously described (Stennicke et al., 1998).

Semiquantitative PCR

For relative quantification of caspase-3 mRNA a reverse transcription (RT)-polymerase chain reaction (PCR) was used with 18sRNA internal control according to the manufacturer's instructions (Ambion; QuantumRNATM 18S internal standard). Total cellular RNA was prepared from 3×10⁶ cells using the RNeasy Kit (Quiagen) and 1 μg total RNA was reverse transcribed with oligo-dT primers and TaqGold polymerase by using the Thermoscript RT-PCR System (Life Technologies). RT product (1 μl) was amplified by caspase-3 specific primers in the presence of 18s ribosomal RNA specific primers and empirically determined ratios of 18s competimers. Amplification was carried out using 1 unit of polymerase in a final volume of 20 μl containing 2.5 mM MgCl₂. TaqGold was activated by incubation at 95° C. for 5 min, and the reactions were cycled 20-37 times at 95° C. for 30 sec, 60 ° C. for 30 sec, and 72° C. for 2 min, followed by a final extension at 72° C. for 10 min. PCR products were visualized on 1% agarose gels stained with ethidium bromide.

Microarray Hybridization and Data Analysis

Total cellular RNA was prepared from untreated and etoposide treated cells after 2 and 4 hours (3×10⁶) using the RNeasy Kit (Quiagen). Labeled cRNA was prepared and hybridized to oligonucleotide microarrays (U95Av2 GeneChip; Affymetrix Incorporated, Santa Clara, Calif.) as described previously (Wodicka et al., 1997), with the exception that the arrays were hybridized at 50° C. for 16-20 h and every experiment was done in duplicates. Scanned image files were analysed with GENECHIP 3.1 (Affymetrix), GeneSpring and Cluster/TreeView. Genes, up- or downregulated in response to etoposide in each of the cell lines, were filtered using the following criteria: maximum hybridization intensity (AD) had to be >200 in at least one sample (untreated, 2 h or 4 h after etoposide treatment), t-test p-value of duplicates lower than 0.2 and increase or decrease in average difference fold of at least 2.0. For each gene the levels of average difference fold change across all cell lines were median-centered, normalized by “Cluster” and output in “TreeView”. Only the genes, which fulfilled the above described three criteria in one of the cell lines (DOHH2, K422 or RL-7), were included.

Plasmids and Sequencing

For sequencing of the coding sequences of caspase-3 and p53 of DOHH2, K422 and RL-7 cells RT-PCRs were performed using gene specific primers and the PCR products were cloned into pCR4-TOPO (Invitrogen).

Results

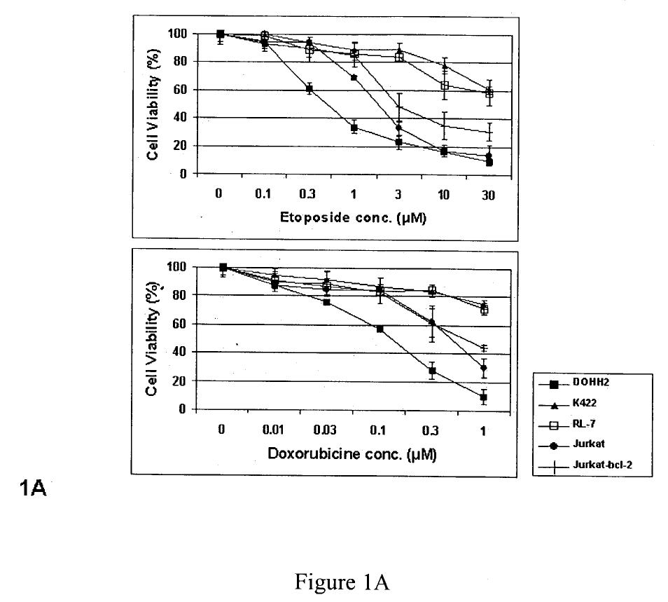

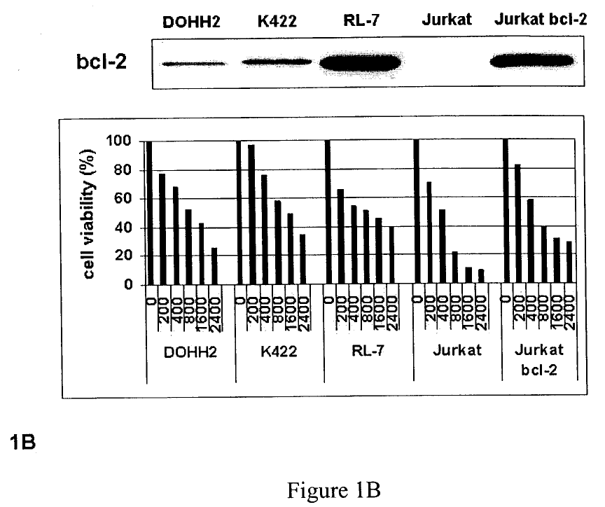

Various human FL cell lines were tested to determine their sensitivity to anticancer drugs. Comparison of the lymphoma cell lines DOHH2, K422 and RL-7 for their response to apoptotic stimuli showed a differential sensitivity to chemotherapeutic drugs. Cells were teseted with the topoisomerase II inhibitor, etoposide, and the anthracyclin, doxorubicin, which have been shown to induce apoptosis by causing DNA double-strand breaks. The K422 and RL-7 cell lines required more than 50-fold higher drug concentrations to obtain a similar 50% reduction in viability (FIG. 1A) in comparison to the DOHH2 cells. Caspase activity assays directly correlated with cell viability in these experiments, indicating that the results did not reflect differences in drug induced cell cycle regulation. The observed drug sensitivities of the three cell lines correlated with the respective clinical histories: K422 and RL-7 cells were derived from chemotherapy-resistant patients.

The apparent IC₅₀ concentrations of etoposide and doxorubicin for inducing cell-death in DOHH2 cells were similar to those observed for Jurkat T-ALL cells (FIG. 1A), which were used as a control. These cells do not express detectable quantities of bcl-2 and are known to be sensitive to most apoptosis stimuli including staurosporine, agonistic anti-Fas antibodies, UV light exposure, gamma irradiation and DNA damaging drugs. Drug response in Jurkat T-cells stably transfected and selected for bcl-2 expression was also evaluated. These cells, which dramatically over express bcl-2 protein, exhibited only a slight enhancement of resistance to DNA damaging drugs in comparison to wild-type Jurkat T-cells.

Bcl-2 Over-Expression in FL Cell Lines is Sufficient to Suppress UV-Induced Cell-Death.

UV-induced apoptosis is reportedly dependent upon mitochondrial release of cyctochrome c, which can be blocked by expression of bcl-2. Cytotoxicity assays following UV light exposure (16-24 hours) were therefore performed to confirm that bcl-2 significantly inhibits the mitochondrial dependent apoptosis pathway in all three FL cell lines (FIG. 1B). UV-induced cytotoxicity inversely correlated with bcl-2 protein levels in all of the cell lines tested. DOHH2, K422, RL-7 and Jurkat T-cells that stably express bcl-2 exhibited similar resistance to UV-induced cell-death, whereas Jurkat T-cells that lack bcl-2 were more sensitive. Thus, bcl-2 expression in chemotherapy sensitive DOHH-2 cells is sufficient for suppressing intrinsic mitochondria dependent cell-death pathways, but not anticancer drug-induced apoptosis. Thus, these DNA damaging drugs and UV exposure engage distinct apoptotic programs in FL cells.

Gene Expression Profiling of FL Cell Lines After Etoposide Treatment.

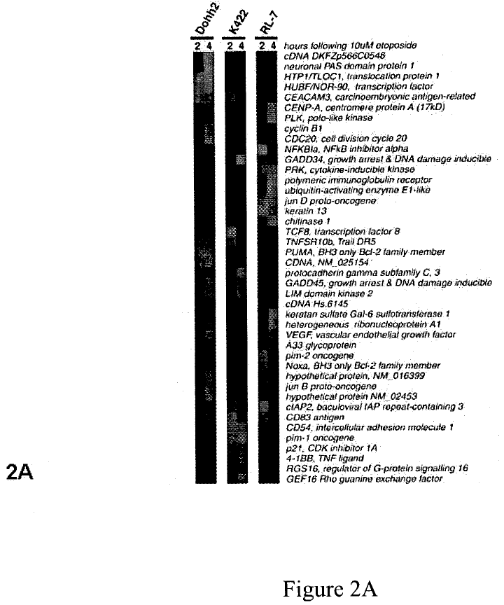

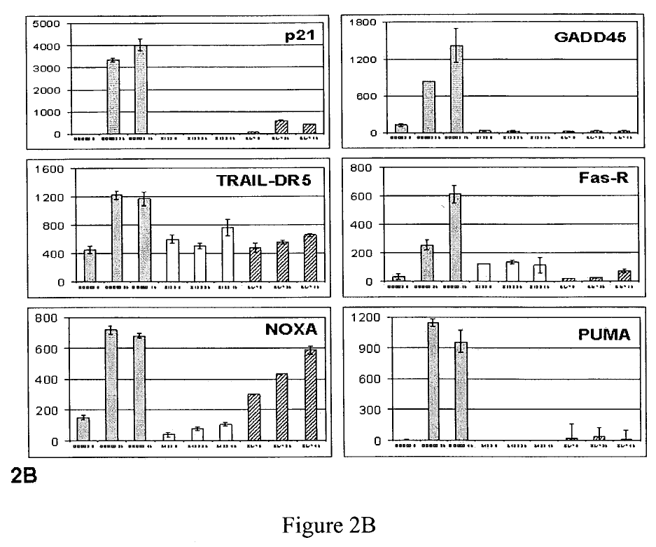

To further characterize the chemosensitive and chemoresistant phenotypes of FL cells after exposure to DNA damaging drugs, oligonucleotide microarrays containing probe sets complementary to 12,533 genes (Affymetrix U95Av2, FIG. 2A) were used to evaluate gene expression. Total RNA was isolated from DOHH2, K422 and RL-7 cells before and after 2 and 4 hours of etoposide treatment. As a primary filter, genes in each cell line that showed a change in RNA levels of at least 2-fold after 2 or 4 hours of etoposide treatment compared to the untreated control (see materials and methods for additional filtering criteria) were searched for. The largest number of genes that exhibited expression changes in response to etoposide was found in the drug sensitive cell line DOHH2 (43 genes in DOHH2, 13 genes in K422 and 12 genes in RL-7) and the vast majority of these (88%) did not change significantly in the chemotherapy resistant cell lines K422 and RL-7. This subset of genes includes the TRAIL death receptor DR5, as well as other genes associated with apoptosis, e.g., the pro-apoptotic bcl-2 family members PUMA and NOXA.

Many of the genes that responded to etoposide treatment in DOHH2 contain promoter sequences with high similarity to the p53 consensus element (5′ PuPuPuC(A/T)(T/A)GpyPyPy-3′ spaced 0-13 base pairs apart), including known p53 regulated genes, such as p21 (Cip-1/WAF1/MDA6), GADD45, DR5, Fas-R, NOXA and PUMA. The raw expression data from several of the known p53-regulated genes showed the magnitude of changes in expression in each of the FL cell lines (FIG. 2B). Very few genes, such as the p53 independent GADD34 and the NF-κB inhibitor alpha (NFκBIα), were commonly upregulated in all three FL cell lines. NFκBIα-mediated inhibition of the transcription factor NF-κB would be predicted to suppress expression of anti-apoptotic genes and thereby sensitize cells to apoptotic stimuli. However, without co-expression of at least some of the above mentioned p53-regulated genes, NFκBIα expression alone was insufficient to facilitate apoptosis

p53 Mutations in Chemotherapy Resistant FL Cell Lines.

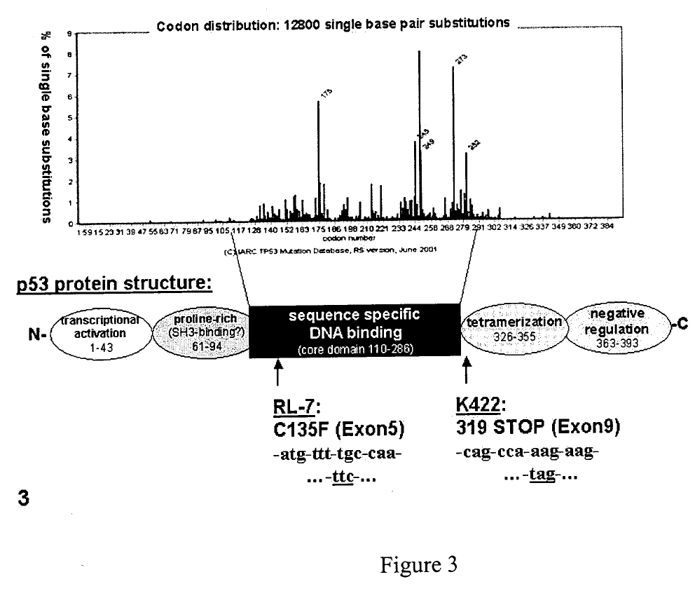

Many of the genes that responded to etoposide in DOHH2 cells were known to be regulated by p53. The p53 cDNA from each of the three FL cell lines was therefore sequenced to determine if p53 mutations contribute to anti-cancer drug resistance in FL. The results showed that DOHH2 cells contain wild type p53, whereas RL=7 cells and K422 cells had mutant forms of p53. The p53 sequence in RL-7 cells had a single base pair substitution in exon 5 of the DNA binding domain (C135F). Comparison with the p53 mutation database of the WHO International Agency for Research on Cancer (Lyon, France) revealed that this cysteine to phenylalanine amino acid exchange is very common in human cancers (FIG. 3). In contrast, the p53 gene in K422 cells contained an unusual pre-terminal stop codon at position 319. The resulting p53 truncation protein, which lacks the tetramerization domain, would be unable to form active tetrameric p53. To date, this mutation, in exon 9 of p53, has only been reported in one Japanese patient with a hepatitis C virus (HCV)-positive hepatocellular carcinoma.

DNA Damaging Drugs Induce Upregulation of the Trail-Dr5 Receptor

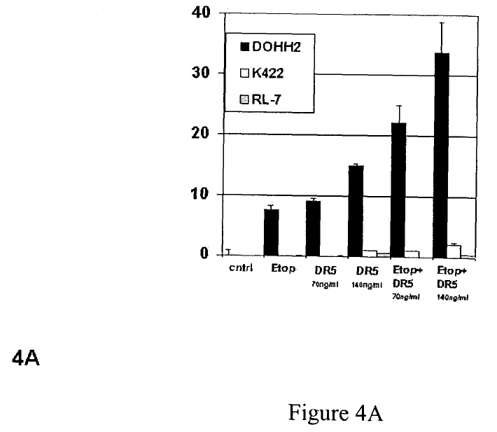

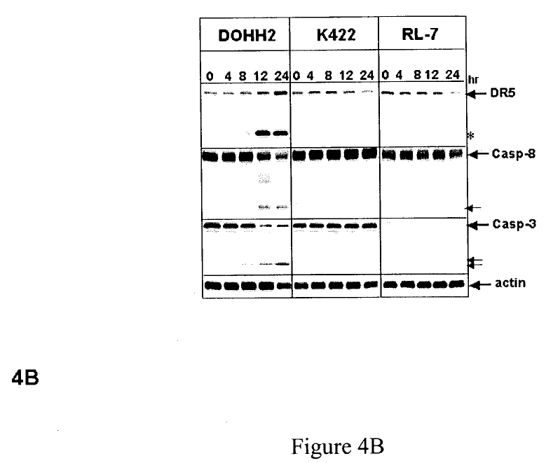

Bcl-2 expressing DOHH2 cells were shown to be sensitive to DNA damaging drugs. Therefore, to test whether DNA damage might engage death receptor pathways that are capable of circumventing bcl-2 through caspase-8-mediated activation of capase-3, and to confirm the gene expression profiling results with TRAIL-DR5 receptor upregulation, it was determined whether engagement of this death receptor would enhance drug-induced cell death in the FL cell lines. DOHH2, K422 and RL-7 cells were treated with etoposide, agonistic anti-DR5 antibodies, or both (FIG. 4A). Pretreatment of DOHH2 cells with etoposide significantly increased apoptosis induced by anti-DR5 antibody compared to drug or anti-DR5 antibody treatment alone, whereas it had a negligible effect in K422 or RL-7 cells. In similar assays, agonistic anti-Fas antibodies had no significant effect on any of these cell lines in the presence or absence of DNA damaging drugs, although the anti-Fas antibodies potently induce apoptosis in other cell lines, such as Jurkat T-cells.

To further characterize the synergistic effect between anti-DR5 antibodies and DNA damaging drugs in DOHH2 cells, protein levels of the components of this death receptor pathway were evaluated. Western blot analysis using antibodies specific for DR5 revealed that following exposure to etoposide (and doxorubicin), DR5 protein levels significantly increased in DOHH2 cells (FIG. 4B). The increased DR5 correlated with pro-caspase-8 activation, which in turn correlated with proteolytic processing and activation of caspase-3 in DOHH2 cells. In contrast, DR5 levels remained constant in K422 and RL-7 cells following exposure to DNA damaging drugs, providing further evidence that mutations of p53 in FL cells suppress activation of this death receptor pathway.

As caspase-8 is the apical caspase in the DR5 pathway; a peptidyl inhibitor of the enzyme was used to further assess the role of the DR5 pathway in responding to DNA damaging drugs. Inhibition of caspase-8 prevented etoposide-induced caspase activation and apoptosis in DOHH2 cells, thus further supporting that DNA damage induced-apoptosis depends upon engagement of the TRAIL-DR5 and caspase-8 pathway.

Drug Resistant RL-7 Cells Suppress Apoptosis by Downregulation of Caspase-3

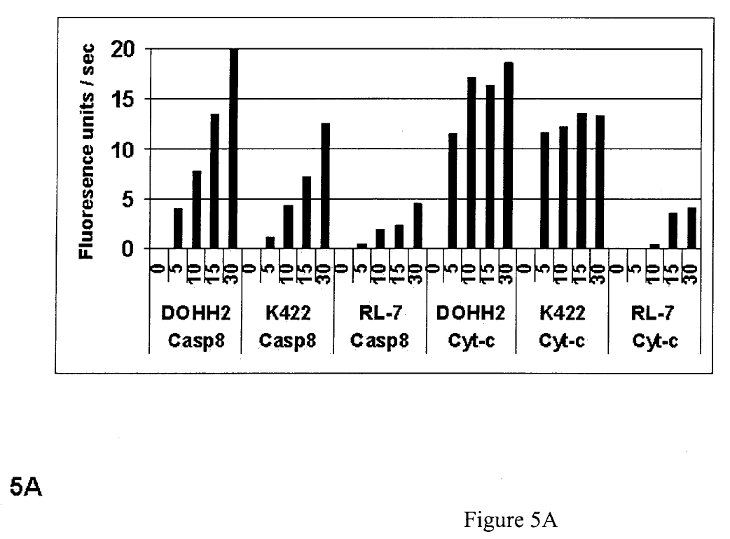

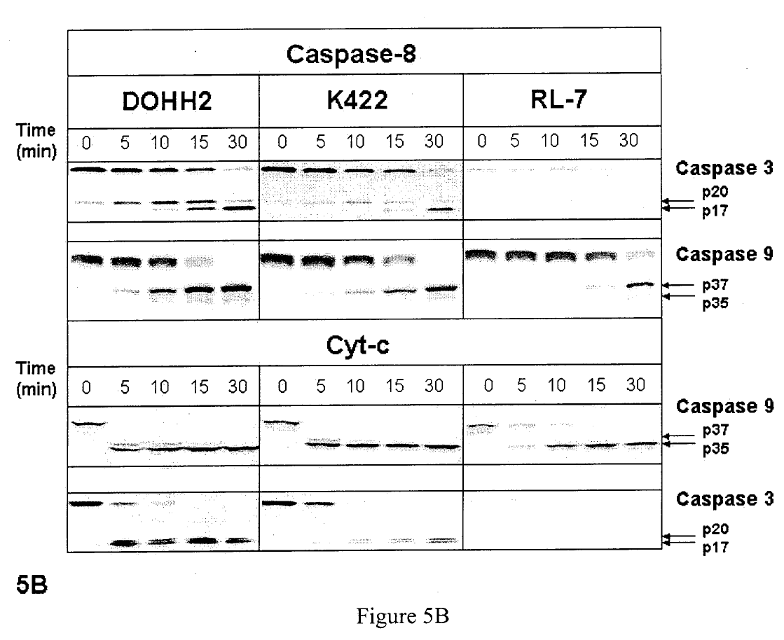

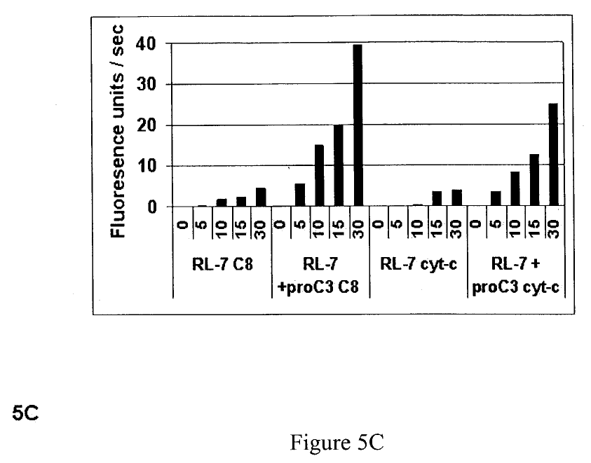

To further characterize defects that contribute to enhanced drug resistance in K422 and RL-7 cells, both the intrinsic (mitochondrial) and extrinsic (death receptor) cell-death pathways were modeled in vitro by addition of cytochrome c or active caspase-8, respectively, to cell-free extracts (Deveraux et al., EMBO J. 17: 2215-2223, 1998.). Direct addition of cytochrome c or caspase-8 to cell lysates should “bypass” the mitochondrial block mediated by bcl-2 in FL cells, as well as the transcriptional defects due to p53 mutation—thereby allowing examination of these prototypical cell-death pathways for further alterations. The results showed that both cyctochrome c and caspase-8 induced significant caspase activity in DOHH2 as well as the chemoresistant K422 cell extracts. However, RL-7 cell extracts exhibited severely reduced caspase activity in response to addition of either protein (FIG. 5A).

To define the biochemical step that is blocked in RL-7 cells, western blotting using caspase-specific antibodies was then used to examine caspase processing and activation induced by addition of cytochrome c or active caspase-8 (FIG. 5B). These data revealed that cytochrome c induced the cleavage of pro-caspase-9 yielding the large p35 subunit characteristic of active caspase-9 in all cell lines; however, the next protease in the intrinsic pathway, caspase 3, is nearly absent in RL-7, whereas DOHH2 and K422 lysates contained ample amounts of pro-caspase-3, which is processed to its active large subunit(s) (˜p20, p 17) following addition of cyctochrome c or active caspase-8 (FIG. 5B). Mimicking the extrinsic pathway through addition of active caspase-8 results in activation of caspase-3, which then can process caspase-9 to the p37 form of the large subunit. Caspase-9 is also processed at later time-points in RL-7 lysate, possibly due to residual caspase-3 activity. Supplementing RL-7 lysates with recombinant pro-caspase-3 resulted in restoration of cytochrome c and caspase-8-induced caspase processing and activation—similar to that observed for endogenous caspase-3 in DOHH2 and K422 lysates (FIG. 5C).

Semiquantitative PCR analysis of caspase-3 mRNA showed reduced transcript levels in RL-7 cells, suggesting that the low pro-caspase-3 protein levels may result from reduction in caspase-3 mRNA levels, rather than translational or posttranslational defects. Sequencing of the caspase-3 DNA from RL-7, as well as K442 and DOHH2 cells, did not identify any mutations in the coding regions of the gene. Thus, RL-7 FL cells, in addition to over-expression of bcl-2 and mutant p53, contain severely reduced levels of pro-caspase-3 protein, thereby providing a further explanation for why these cells are extremely resistant to both death receptor as well as mitochondria dependent apoptosis programs.

High-Throughput Differential Cytotoxicity Screen for Small Molecules that Induce Alternative Cell-Death Pathways

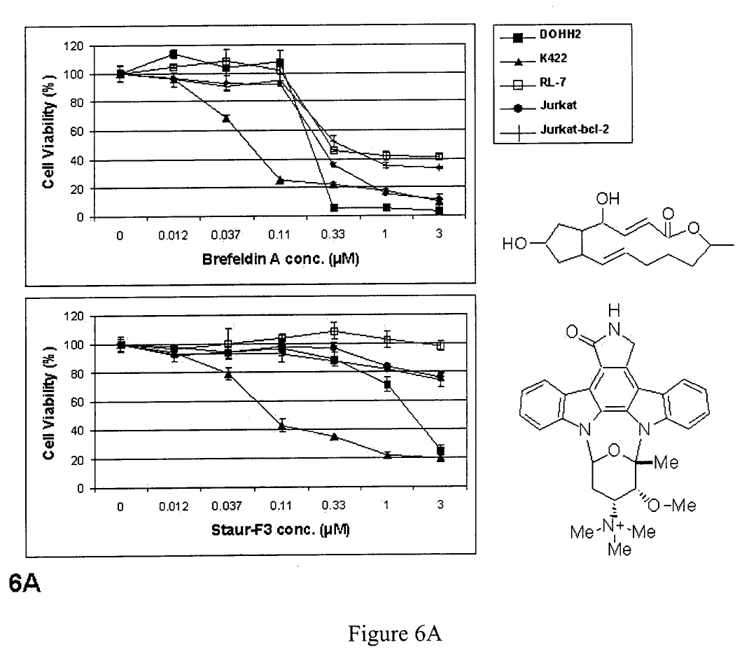

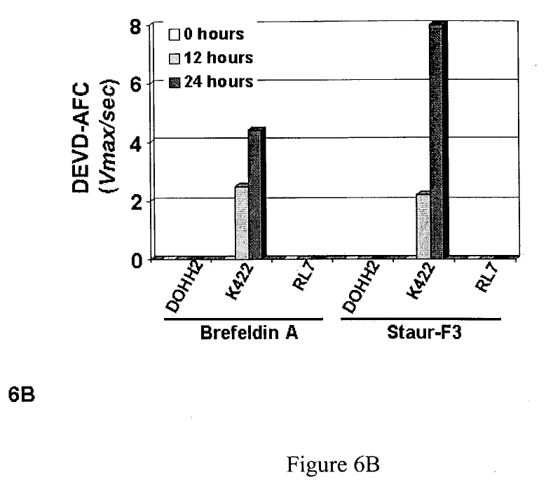

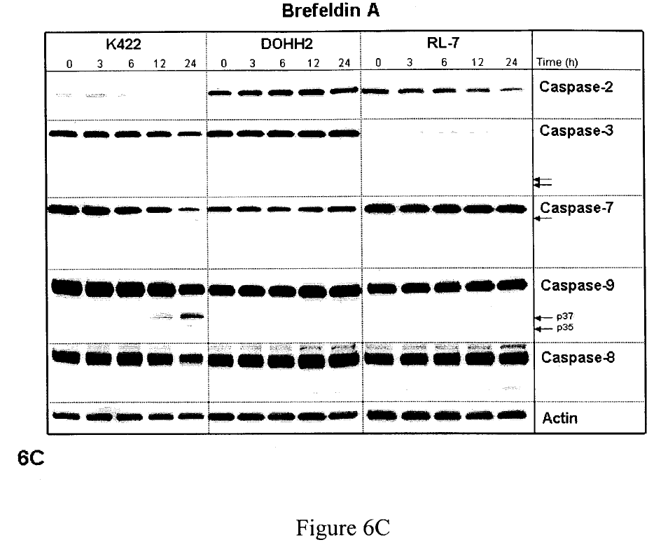

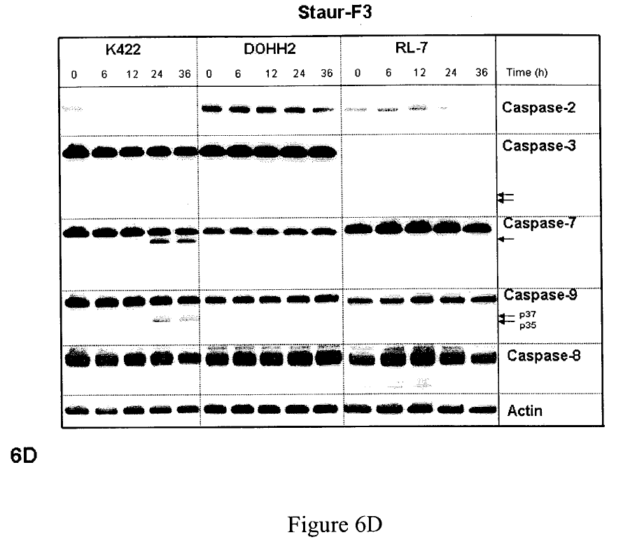

As described herein, the K422, RL-7, and DOHH2 FL cell lines exhibited distinct responses to DNA damaging drugs. These differences were exploited to screen for compounds with alternative cell-death inducing mechanisms. Small molecule libraries were screened in a high-throughput format for compounds that significantly decreased the viability of K422 or RL-7, but not DOHH2 cells (for additional information see materials and methods). Two compounds that exhibited preferential cytotoxicity in the K422 cell line were identified (FIG. 6A), a known and commercially available compound, Brefeldin A, and a Staurosporine derative, Staur-F3, the structure of which is shown below. Brefeldin A and the Staurosporine derivative exhibited a 6-fold and 25-fold difference, respectively, in their IC₅₀ concentrations in K422 cells compared to the other cell lines tested.

Staurosporine Derivative:

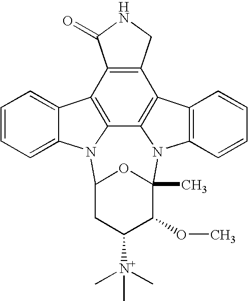

The staurosporine analog is described, e.g., in Caravatti et al., Bioorganic & Medicinal Chemistry Letters 4:399-404, 1994. Derivatives, e.g., acylated derivatives are known, see, e.g., U.S. Pat. No. 5,093,330. Such derivatives may be used to induce apoptosis.

Morphological examination of FL cells treated with Brefeldin A or the Staurosporine analog showed that only K422 exhibited the classical features of apoptosis, i.e., characteristics such a cell condensation, membrane blebbing and eventual fragmentation. To quantify this response, treated and untreated cells were lysed and assayed for caspase activity (FIG. 6B). Addition of 100 nM Brefeldin A or the Staurosporine analog induced caspase activation and western blot analysis of these samples showed caspase processing of the caspases-2, -3, -7 and -9 only in K422 but not DOHH2 or the caspase-3 deficient RL-7 cells (FIG. 6C, D). Processing of caspase-9 is likely due to caspase-3 or caspase-7, but not autocatalytic activation through the intrinsic pathway, as the p37 form of the caspase-9 large subunit, rather than the p35 form, was primarily observed. The death receptor-associated caspase-8 remained unprocessed in all of the cell lines. Therefore, both the intrinsic and extrinsic pathways remain blocked in K422, and these compounds appear to activate the executioner caspase-3 and -7 through alternative means. Interestingly, similar caspase processing was observed for both compounds indicating that these agents induce apoptosis through a common pathway. Caspase-2 is possibly the apical caspase in this alternative pathway since Brefeldin A is a potent inducer of endoplasmic reticulum stress by disassembly of the Golgi complex, which reportedly can facilitate local caspase-2 activation (Chardin & McCormick, Cell 97:153-5, 1999; Mancini et al., J Cell Biol. 149:603-12, 2000; and Ferri & Kroeme,r Nat Cell Biol. 3:E255-63, 2001).

### SUMMARY

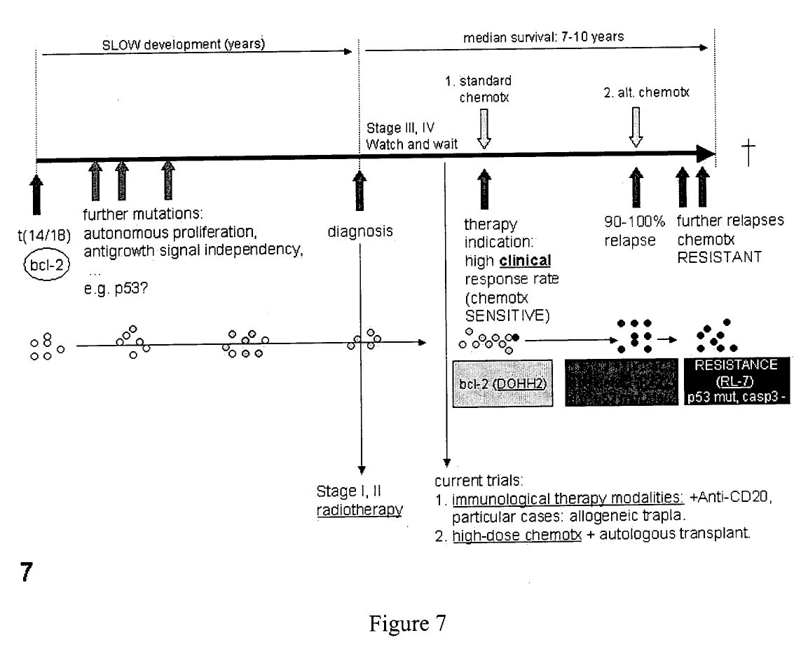

Due to bcl-2 over-expression, FL is a slow growing, indolent lymphoma that is incurable with standard chemotherapy. Predisposed by the mitochondrial cell-death block through bcl-2, it is very likely that anticancer drugs select for FL subpopulations containing mutations in the TRAIL-DR5 cell-death pathway e.g. p53 and/or caspase-3 (see, FIG. 7). Consistent with the idea of persistence of drug-surviving lymphoma cells is the observation that standard chemotherapy is rarely capable of generating a molecular response.

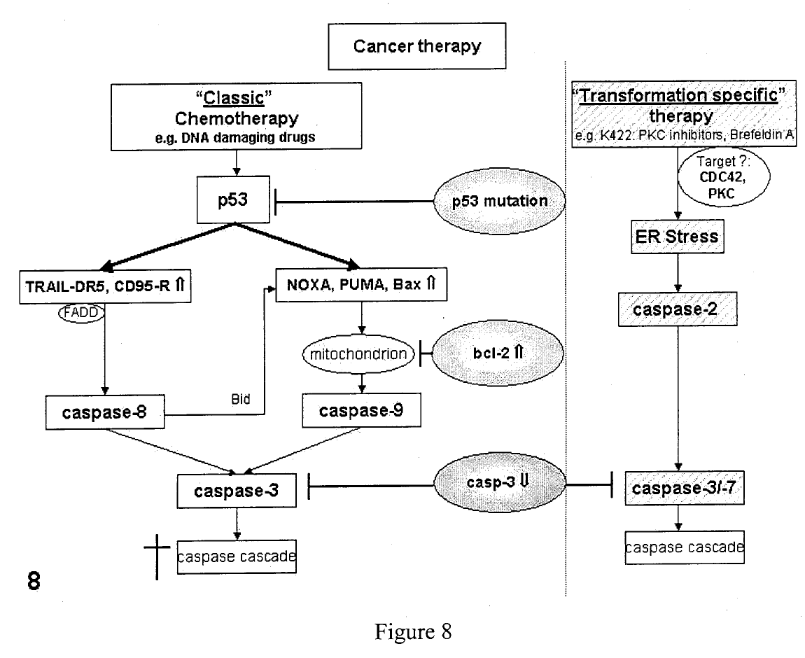

Models of “classic” and “transformation/survival specific” chemotherapy induced cell-death pathways are shown in FIG. 8. Although follicular lymphoma cells over express the anti-apoptotic protein bcl-2 they are initially sensitive to anticancer therapy. The reason for this ostensible paradox is that DNA damaging drugs not only induce pro-apoptotic bcl-2 family members such as NOXA and PUMA, but also the TRAIL-DR5 death receptor, which activates caspase-8 and caspase-3 in a bcl-2 independent manner. The common upstream regulator of both apoptosis initiation pathways is the transcription factor p53. In DNA damaging drug resistant FL cells this death receptor apoptosis pathway is severely compromised by mutational inactivation of p53-mediated transcription or repression of caspase-3 expression. Nevertheless there is the possibility to induce alternative programmed cell-death pathways in chemotherapy resistant tumor cells by targeting cell specific transformation or survival molecules as show for Brefeldin A and the potential protein kinase inhibitor Staur-F3, which induced p53 and bcl-2 independent apoptosis in K422 cells in concentrations nontoxic to normal or non-target bearing tumor cells.

Thus, employing a selective screening strategy, a known drug, Brefeldin A, and a staurosporine analog (Staur-F3) were identified that elicit a similar cell-death program. Brefeldin A is known to be a specific “non-competitive” inhibitor of the GTP binding protein ARF-1, which is required for Golgi complex formation (Chardin & McCormick, supra). Treatment with Brefeldin A induces endoplasmatic reticulum (ER) stress, which has recently been implicated as a novel alternative apoptosis pathway (Rao et al, J Biol. Chem. 277:21836-42, 2002). Interestingly, a review of the NCI database on the NCI 60 tumor cell panel sensitivity to Brefeldin A reveals that one of the most sensitive cell lines is NCI/ADR (data available at http://.nci.nih.gov). These cells, like K422, are resistant to doxorubicin (adriamycin).

The staurosporine derivative can function as a protein kinase inhibitor (e.g., PKC) and therefore implicates kinases in the ER/Golgi apoptosis pathway.

In summary, cell lines established from chemosensitive and resistant patients were employed to further define drug induced cell-death pathways and identify alterations in these pathways that contribute to chemoresistance. The cells were used to screen for compounds with cell-death inducing mechanisms distinct from DNA damaging drugs like etoposide and doxorubicin.

### Example 2

### Identification of Proteins That Modulate TRAIL-Mediated Apoptosis

This Example provides a list of therapeutic targets for modulation of the TRAIL-DR5 death receptor pathway. Small interfering RNA (siRNA) molecules for each of the genes listed in Table 1 were synthesized and separately introduced into cells. The effect on DR5-mediated induction of the TRAIL-DR5 death receptor pathway was then determined. In each case, disrupting expression of the gene enhanced TRAIL-mediated cell death. Accordingly, the listed genes are suitable targets for use in screening assays to identify compounds that stimulate apoptosis and therefore are useful in treating cancer.

A screening assay may comprise testing a candidate agent for the ability to reduce activity of a target polypeptide listed in Table 1. Activity can be assessed either directly or indirectly. For example, activity can be measured using known assays, e.g., enzymatic assays, for the various target polypeptides. Alternatively, activity can be assessed by measuring levels of expression of either mRNA or protein in a cell that expressed the target polypeptide, as described herein.

A candidate agent can also be tested using a reporter construct that comprises a polynucleotide that encodes a reporter polypeptide operably linked to a polynucleotide that comprises a regulatory region from the gene that encodes the target polypeptide. Such assays are well known in the art. Basic texts disclosing exemplary methods include Sambrook and Russell, supra, and Ausubel, supra.

Either cellular or solid-state assays may be used in screening for inhibitors that target the proteins in Table 1. Often, screening assays for inhibitors of the target polypeptides are performed using cell-based assays. The cells can express the protein endogenously or can be cells transfected with an expression vector comprising a nucleic acid sequence encoding the target polypeptide, or active fragment of the target polypeptide, or a reporter construct. In vitro assays may also be used to screen for inhibitors. Often, the polypeptides used in such assays are recombinantly produced.

Screening assays for inhibitors of the target polypeptides can be performed manually or using a high throughput format.

TABLE 1
PHOSPHATIDYLINOSITOL-KINASE-RELATED
H.s. mitogen-activated protein kinase kinase kinase 5 (MAP3K5), mRNA‘’‘’‘’‘’‘’‘’‘’
JIK: STE20-like kinase
H.s MAP kinase-interacting serine/threonine kinase 1 (MKNK1), mRNA‘’‘’‘’
Human mRNA for KIAA0930 protein, partial cds.
H.s mitogen-activated protein kinase-activated protein kinase 2 (MAPKAPK2), transcript variant 1,
mRNA‘’
PHOSPHATIDYLINOSITOL-KINASE-RELATED
H.s mitogen-activated protein kinase kinase 5 (MAP2K5),
Q03533 SERINE/THREONINE PROTEIN KINASE
Q62862 SER/THR FAMILY OF PROTEIN KINASES-RELATED
H.s. cyclin-dependent kinase 6 (CDK6), mRNA‘’‘’‘’‘’‘’‘’‘’
H.s. activin A receptor type II-like 1 (ACVRL1), mRNA‘’‘’‘’‘’‘’‘’‘’
H.s. Gardner-Rasheed feline sarcoma viral (v-fgr) oncogene homolog (FGR), mRNA‘’‘’‘’‘’‘’‘’
Human TGF beta inducible nuclear protein TINP1 (TINP1) mRNA, complete cds.
H.s muscle, skeletal, receptor tyrosine kinase (MUSK), mRNA‘’‘’‘’
Human CGI-53 protein mRNA, complete cds.
H.s budding uninhibited by benzimidazoles 1 (yeast homolog) (BUB1), mRNA‘’‘’‘’
H.s ribosomal protein S6 kinase, 90 kD, polypeptide 5 (RPS6KA5), mRNA‘’‘’‘’
H.s. CDC-like kinase 2 (CLK2), transcript variant phclk2, mRNA‘’‘’‘’‘’‘’‘’‘’
2117904 RIBOSE-PHOSPHATE PYROPHOSPHOKINASE-RELATED
H.s v-yes-1 Yamaguchi sarcoma viral related oncogene homolog (LYN), mRNA‘’‘’‘’
MAPK7: mitogen-activated protein kinase 7
H.s. p21/Cdc42/Rac1-activated kinase 1 (yeast Ste20-related) (PAK1), mRNA‘’‘’‘’‘’‘’‘’‘’
H.s v-akt murine thymoma viral oncogene homolog 1 (AKT1), mRNA‘’‘’‘’
MAPK9: mitogen-activated protein kinase 9
H.s. mitogen-activated protein kinase kinase kinase kinase 4 (MAP4K4), mRNA‘’‘’‘’‘’‘’‘’‘’
H.s MEK kinase 1 (MEKK1) mRNA, partial cds‘’‘’‘’
Human CGI-06 protein mRNA, complete cds.
H.s GTPase regulator associated with FAK
Homo sapiens nemo-like kinase (LOC51701), mRNA.
H.s CHK1 (checkpoint, S. pombe) homolog (CHEK1), mRNA‘’‘’‘’
H.s. tousled-like kinase 1 (TLK1), mRNA‘’‘’‘’‘’‘’‘’‘’
CALM3: calmodulin 3 (phosphorylase kinase, delta)
pre-B-cell leukemia transcription factor 1
Human cDNA FLJ20594 fis, clone KAT08731.
Human, Similar to CG8405 gene product, clone MGC: 4022, mRNA, complete cds.
unknown PROTEIN KINASE
MAP PROTEIN KINASE-RELATED
moderately similar to HYPOTHETICAL 47.6 KD PROTEIN IN CHROMOSOME III.
H.s. p21 (CDKN1A)-activated kinase 4 (PAK4), mRNA‘’‘’‘’‘’‘’‘’‘’
H.s. v-erb-b2 avian erythroblastic leukemia viral oncogene homolog 3 (ERBB3), mRNA‘’‘’
Human embryonic lung protein (HUEL) mRNA, complete cds.
H.s glycogen synthase kinase 3 alpha (GSK3A), mRNA‘’‘’‘’
DR4 trail receptor 1
Bid
Novel Human gene mapping to chomosome 22.
H.s B lymphoid tyrosine kinase (BLK), mRNA‘’‘’‘’
Caspase-8
Apaf-1
Fadd

All publications and patent applications cited in this specification are herein incorporated by reference as if each individual publication or patent application were specifically and individually indicated to be incorporated by reference.

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or scope of the appended claims.

What is claimed is:

1. A method of identifying a compound that is selectively cytotoxic to chemoresistant cancer cells, the method comprising:
a) contacting a candidate compound with cancer cells that have a defect in a TRAIL-DR5 death receptor pathway;
b) contacting the candidate compound with normal cells;
c) determining the level of cell death in the cancer cells and in the normal cells; and
d) selecting a candidate compound that increases cell death in the cancer cells relative to normal cells, thereby identifying a compound that is selectively cytotoxic to chemoresistant cancer cells.

2. The method of claim 1, wherein the chemoresistant cancer cells are non-Hodgkins lymphoma cells.

3. The method of claim 2, wherein the chemoresistant cancer cells are follicular lymphoma cells.

4. The method of claim 3, wherein the cells are K422 cells or RL-7 cells.

5. The method of claim 1, wherein the chemoresistant cancer cells are resistant to DNA damaging agents.

6. The method of claim 1, wherein the cancer cells overexpress bcl-2.

7. The method of claim 1, wherein the defect in the TRAIL-DR5 death receptor pathway is a defect in p53.

8. The method of claim 1, wherein the defect in the TRAIL-DR5 death receptor pathway is a defect in caspase 3.

9. The method of claim 1, wherein the cancer cells have a defect in p53 and in caspase 3.

10. The method of claim 1, wherein the step of contacting the normal cells with the candidate compound is performed before selecting the compound.

11. The method of claim 1, wherein the candidate compound is a member of a library of candidate compounds that are being tested for inhibiting proliferation of the cancer cells, but not the normal cells.

12. A method of screening for a compound that is selectively cytotoxic to a subset of chemoresistant lymphomas, the method comprising:
providing a first cell population from a first lymphoma that has a first defect in a TRAIL DR5 apoptotic pathway;
contacting the first cell population with a candidate compound;
detecting the level of cell death in the first population of cells;
providing a second cell population from a second chemoresistant lymphoma that has a second defect in the TRAIL DR5 pathway;
contacting the second cell population with the candidate compound;
detecting the level of cell death in the second population of cells; and
selecting a compound that increases cell death in only one of the cell populations, thereby obtaining a compound that is selective for the first or second subset of chemoresistant lymphomas.

13. The method of claim 12, wherein the lymphoma has increased levels of bcl-2.

14. The method of claim 12, wherein the lymphoma is a non-Hodgkin's lymphoma.

15. The method of claim 14, wherein the lymphoma is a follicular lymphoma.

16. The method of claim 12, wherein the first defect is a defect in p53.

17. The method of claim 16, wherein the defect in p53 is an absence of a tetramerization domain or a substitution of Cys135 with a different amino acid.

18. The method of claim 12, wherein the second defect is a defect in caspase 3.

19. The method of claim 12, wherein the first population of cells comprises K422 cells and the second population comprises RL-7 cells.

20. A method of killing a cancer cell, the method comprising administering a compound selected as set forth in claim 1 or claim 12.

21. A method of killing a chemosresistant lymphoma cell having a defect in p53, the method comprising administering an effective amount of staurosporine analog.

22. The method of claim 21, wherein the lymphoma is a non-Hodgkin's lymphoma.

23. The method of claim 22, wherein the non-Hodgkin's lymphoma is follicular lymphoma.

24. A method for treating a cancer patient, the method comprising:
a) testing cancer cells from a cancer patient for a defect in a TRAIL-DR5 death receptor pathway; and
b) if cells exhibit defect in the TRAIL-DR5 death receptor pathway, treating the cancer with a therapeutic agent for which induction of DNA damage is not the therapeutic agent's primary mechanism of action.

25. The method of claim 24, wherein the cancer cells overexpress bcl-2.

26. The method of claim 24, wherein the defect is a p53 defect.

27. The method of claim 26, wherein the p53 defect is detected using an antibody that binds to p53.

28. The method of claim 24, wherein the TRAIL-DR5 death receptor pathway defect is characterized by a reduced level of caspase 3.

29. The method of claim 24, wherein the therapeutic agent is an apoptosis-inducing agent.

30. The method of claim 24, wherein the therapeutic agent is selected from the group consisting of an anti-CD20 antibody, an allogeneic T-lymphocyte, a bcl-2 inhibitor, an agonistic anti-TRAIL antibody, and a TRAIL receptor ligand.

31. The method of claim 30, wherein the therapeutic agent is an agonistic anti-DR5 antibody or a ligand for DR5.

32. The method of claim 31, wherein the therapeutic agent is rituximab or TRAIL/Apo2L.

33. The method of claim 24, wherein the cancer is a lymphoma.

34. The method of claim 33, wherein the lymphoma is a non-Hodgkin's lymphoma.

35. The method of claim 34, wherein the non-Hodgkin's lymphoma is follicular lymphoma.

36. The method of claim 24, wherein the cancer patient is treated with a chemotherapeutic DNA damaging agent prior to testing for the TRAIL-DR5 death receptor pathway defect.

37. The method of claim 24, wherein treatment of the cancer patient with the DNA damaging agent is discontinued if a TRAIL-DR5 death receptor pathway defect is detected.

38. The method of claim 24, wherein the DNA damaging agent is an alkylating agent or a topoisomerase II inhibitor.

39. A method for monitoring a chemotherapeutic treatment of a cancer patient, the method comprising testing cancer cells obtained from the patient periodically during a cancer treatment for a defect in a TRAIL-DR5 death receptor pathway, wherein, when an increase in the prevalence of cancer cells having the defect occurs, the method further comprises treating the patient with a therapeutic agent for which induction of DNA damage is not the therapeutic agent's primary mechanism of action.

40. The method of claim 39, wherein the cancer is a lymphoma.

41. The method of claim 39, wherein the defect is a p53 defect.

42. The method of claim 39, wherein the TRAIL-DR5 death receptor pathway defect is characterized by a reduced level of caspase 3.

43. A method for identifying an agent that inhibits proliferation of cancer cells, the method comprising testing a candidate agent for ability to reduce activity of a target polypeptide encoded by a gene listed in Table 1, wherein a candidate agent that reduces activity of the target polypeptide is useful to inhibit proliferation of the cancer cells.

44. The method of claim 43, wherein the agent kills the cancer cells.

45. The method of claim 44, wherein the agent kills the cancer cells by inducing apoptosis.

46. The method of claim 43, wherein the candidate agent is tested for ability to inhibit enzymatic activity of the target polypeptide.

47. The method of claim 43, wherein the candidate agent is tested for ability to decrease expression of a gene that encodes the target polypeptide.

48. The method of claim 47, wherein the candidate agent is tested by:
a) providing a cell that comprises a reporter construct, wherein the reporter construct comprises a polynucleotide that encodes a reporter polypeptide operably linked to a polynucleotide that comprises a regulatory region obtained from the gene that encodes the target polypeptide;
b) contacting the cell with the candidate agent; and
c) determining whether expression of the reporter construct is decreased in the presence of the candidate agent compared to expression of the reporter construct in the absence of the candidate agent.

49. The method of claim 47, wherein the candidate agent is tested by contacting the candidate agent with cells that comprise a gene that encodes the target polypeptide and detecting a decrease in mRNA that encodes the target polypeptide in cells exposed to the candidate agent compared to cells not exposed to the candidate agent.

50. The method of claim 49, wherein the method comprises hybridizing nucleic acids obtained from the cell to a probe that has a nucleotide sequence that is complementary to a nucleic acid that encodes the target gene.

51. A method for identifying a gene that is involved in a cell death pathway, the method comprising:
a) providing a library of cDNA or inhibitory RNA molecules, wherein each library member is present in a cell;
b) contacting the cells with a compound that can modulate cell death in a cell that does not comprise the cDNA or inhibitory RNA molecules; and
c) identifying library members in which the compound modulates cell death differently than in a cell that does not contain the cDNA or inhibitory RNA molecule.

52. The method of claim 51, wherein the inhibitory RNA molecules are small interfering RNA (siRNA) molecules.

53. The method of claim 51, wherein the cells are lymphoma cells.
